(19) United States
(12) Patent Application Publication
(10) Pub. No.: US 2014/0314857 A1
(43) Pub. Date: Oct. 23, 2014

(54) FORMULATIONS FOR ENHANCED BIOAVAILABILITY OF ZANAMIVIR

(71) Applicant: ALA WAI PHARMA INC., Honolulu, HI (US)

(72) Inventors: Eric Holmes, Bothell, WA (US); Michael Hite, Seattle, WA (US); Gary K. Ostrander, Honolulu, HI (US)

(21) Appl. No.: 14/320,337

(22) Filed: Jun. 30, 2014

Related U.S. Application Data
(63) Continuation of application No. PCT/US2013/020074, filed on Jan. 3, 2013.
(60) Provisional application No. 61/583,526, filed on Jan. 5, 2012.

(51) Int. Cl. A61K 47/22; A61K 47/18; A61K 47/20; A61K 31/351; A61K 47/10

(52) U.S. Cl. CPC A61K 47/22; A61K 31/351; A61K 47/10; A61K 47/20; A61K 47/183
USPC 424/490; 514/459

(57) ABSTRACT

In accordance with the present invention, there are provided compositions comprising zanamivir and at least one permeability enhancer. The compositions can increase the amount of zanamivir capable of being transported across a cell membrane (such as a Caco-2 cell membrane), and can increase this amount by at least 150% relative to the amount capable of being transported across the cell membrane in the absence of the permeability enhancer. Also provided are oral dosage forms of the compositions, which comprise a therapeutically effective amount of zanamivir and a permeability-enhancing amount of a permeability enhancer. The oral dosage forms can further comprise an enteric- or pH-sensitive coating or layer surrounding the composition. Also provided in accordance with the present invention are methods for treating or preventing influenza infection.

### RELATED APPLICATIONS

This application is a continuation of International (PCT) Application No. PCT/US2013/020074, filed on Jan. 3, 2013, which claims priority from U.S. Provisional Patent Application No. 61/583,526, filed Jan. 5, 2012, each of which is hereby incorporated by reference in its entirety and for all purposes.

### FIELD OF THE INVENTION

The invention relates to enhancing the permeability and bioavailability of polar active agents such as zanamivir.

### BACKGROUND OF THE INVENTION

Zanamivir is a member of a class of antiviral agents that work by inhibiting viral neuraminidase, an enzyme essential for the influenza virus to replicate and infect its hosts. In addition to influenza A and B, avian influenza virus (H5N1) has been shown to be sensitive to zanamivir. However, animal studies with oral forms of zanamivir have demonstrated very poor oral bioavailability thereof.

Accordingly there is a need for neuraminidase inhibitor compositions which exhibit improved bioavailability and efficacy when administered orally for treatment or prevention of a variety of indications, e.g., influenza infections.

### SUMMARY OF THE INVENTION

The invention features compositions comprising zanamivir and at least one permeability enhancer. The compositions can increase the amount of zanamivir capable of being transported across a cell membrane (such as a Caco-2 cell membrane), and can increase this amount by at least 150% relative to the amount capable of being transported across the cell membrane in the absence of the permeability enhancer.

Suitable permeability enhancers for use in the practice of the present invention include fatty acids, fatty acid esters, fatty acid salts, glycerol, glycerol monocaprylate, surfactants, cyclodextrins, sodium salicylate, ethylenediamine tetraacetic acid, citric acid, chitosan, chitosan derivatives, N-trimethyl chitosan chloride, monocarboxymethyl-chitosan, palmitoyl carnitine chloride, acyl carnitines, ethylene glycol tetraacetic acid, 3-alkylamido-2-alkoxypropyl-phosphocholine derivatives, dimethylpalmityl-ammonio propanesulfonate, alkanoylcholines, N-acetylated amino acids, mucoadhesive polymers, phospholipids, piperine, 1-methylpiperazine, α-amino acids, mineral oil, or the like.

In accordance with the present invention, there are also provided oral dosage forms of the compositions, which comprise a therapeutically effective amount of zanamivir and a permeability-enhancing amount of a permeability enhancer. The oral dosage forms can further comprise an enteric- or pH-sensitive coating or layer surrounding the composition. In the oral dosage forms, the permeability enhancer can be glycerol, glycerol monocaprylate, dimethylpalmityl-ammonio propanesulfonate, and the like.

The permeability enhancer can be present in the composition at a concentration from about 0.1 wt % to about 99 wt %, based on the combined weight of the zanamivir and the permeability enhancer.

Also provided in accordance with the present invention are methods for treating or preventing influenza infection. Generally, the methods comprise administering to a subject in need thereof a composition comprising zanamivir and at least one permeability enhancer, including oral dosage forms of such compositions. In the compositions employed in accordance with the invention methods, the permeability enhancer can be glycerol, glycerol monocaprylate, dimethylpalmityl-ammonio propanesulfonate, and the like.

### BRIEF DESCRIPTION OF THE DRAWINGS

FIG. 1 shows the individual plasma concentration of zanamivir versus time following intravenous administration in male Sprague-Dawley rats at 1.5 mg/animal in normal saline (see Example 5). Diamonds refer to test rat #951, squares refer to test rat #952 and triangles refer to test rat #953.

FIG. 2 shows the average plasma concentration of zanamivir versus time following intravenous administration in male Sprague-Dawley rats at 1.5 mg/animal from normal saline (see Example 5).

FIG. 3 shows the individual plasma concentration of zanamivir versus time following intraduodenal administration in male Sprague-Dawley rats at 1.5 mg/animal from a Capmul MCM L8 formulation (see Example 5). Diamonds refer to test rat #9545, squares refer to test rat #955 and triangles refer to test rat #956.

FIG. 4 shows the average plasma concentration of zanamivir versus time following intraduodenal administration in male Sprague-Dawley rats at 1.5 mg/animal from a Capmul MCM L8 formulation (see Example 5).

FIG. 5 shows the individual plasma concentration of zanamivir versus time following intraduodenal administration in male Sprague-Dawley rats at 1.5 mg/animal from glycerol formulation (see Example 5). Diamonds refer to test rat #957, squares refer to test rat #958 and triangles refer to test rat #959.

FIG. 6 shows the average plasma concentration of zanamivir versus time following intraduodenal administration in male Sprague-Dawley rats at 1.5 mg/animal from glycerol formulation (see Example 5).

FIG. 7 shows the individual plasma concentration of zanamivir versus time following intraduodenal administration in male Sprague-Dawley rats at 1.5 mg/animal from PBS formulation (see Example 5). Diamonds refer to test rat #960, squares refer to test rat #961 and triangles refer to test rat #962.

FIG. 8 shows the average plasma concentration of zanamivir versus time following intraduodenal administration in male Sprague-Dawley rats at 1.5 mg/animal from PBS formulation (see Example 5).

FIG. 9 shows the individual plasma concentration of zanamivir versus time following intraduodenal administration in male Sprague-Dawley rats at 1.5 mg/animal from PBS formulation (glycerol, −2 hr pre-dose—see Example 5). Diamonds refer to test rat #963, squares refer to test rat #964 and triangles refer to test rat #965.

FIG. 10 shows the average plasma concentration of zanamivir versus time following intraduodenal administration in male Sprague-Dawley rats at 1.5 mg/animal from PBS formulation (glycerol, −2 hr pre-dose—see Example 5).

FIG. 11 shows the average plasma concentration of zanamivir versus time following intraduodenal administration in male Sprague-Dawley rats at 1.5 mg/animal from several different formulations (diamonds refer to Capmul MCM L8 formulations; squares refer to glycerol formulations; triangles refer to PBS formulations; and circles refer to PBS formulations with glycerol pre-treatment at −2 hrs; see Example 5).

FIG. 12 provides a comparison of the bioavailability of zanamavir from different formulations after intraduodenal administration in male Sprague-Dawley rats at 1.5 mg/animal.

FIG. 13 shows the individual plasma concentration of zanamivir versus time following intraduodenal administration in male Sprague-Dawley rats at 1.5 mg/animal from glycerol (100 μL) formulation (see Example 6). Diamonds refer to test rat #181, squares refer to test rat #182 and triangles refer to test rat #183.

FIG. 14 shows the average plasma concentration of zanamivir versus time following intraduodenal administration in male Sprague-Dawley rats at 1.5 mg/animal from glycerol (100 μL) formulation (see Example 6).

FIG. 15 shows the individual plasma concentration of zanamivir versus time following intraduodenal administration in male Sprague-Dawley rats at 1.5 mg/animal from glycerol (150 mL) formulation (see Example 6). Diamonds refer to test rat #184, squares refer to test rat #185 and triangles refer to test rat #186.

FIG. 16 shows the average plasma concentration of zanamivir versus time following intraduodenal administration in male Sprague-Dawley rats at 1.5 mg/animal from glycerol (150 μL) formulation (see Example 6).

FIG. 17 shows the individual plasma concentration of zanamivir versus time following intraduodenal administration in male Sprague-Dawley rats at 1.5 mg/animal from PBS (10 μL) formulation (150 μL glycerol, −2 hr pre-dose—see Example 6). Diamonds refer to test rat #187, squares refer to test rat #188 and triangles refer to test rat #189.

FIG. 18 shows the average plasma concentration of zanamivir versus time following intraduodenal administration in male Sprague-Dawley rats at 1.5 mg/animal from PBS (50 mL) formulation (150 μL glycerol, −2 hr pre-dose—see Example 6).

FIG. 19 shows the individual plasma concentration of zanamivir versus time following intraduodenal administration in male Sprague-Dawley rats at 1.5 mg/animal from PBS (50 mL) formulation (50 μL Capmul MCM L8, −2 hr pre-dose—see Example 6). Diamonds refer to test rat #190, squares refer to test rat #191 and triangles refer to test rat #192.

FIG. 20 shows the average plasma concentration of zanamivir versus time following intraduodenal administration in male Sprague-Dawley rats at 1.5 mg/animal from PBS (50 mL) formulation (50 μL Capmul MCM L8, −2 hr pre-dose—see Example 6).

FIG. 21 shows the average plasma concentration of zanamivir versus time following intraduodenal administration in male Sprague-Dawley rats at 1.5 mg/animal from different formulations (diamonds refer to 100 μL glycerol; squares refer to 150 μL glycerol; blackened triangles refer to 50 μL PBS after 150 μL pretreatment with glycerol at −2 hr; and open triangles refer to 50 μL PBS after 50 μL pretreatment with Capmul MC L8 at −2 hr—see Example 6).

FIG. 22 shows the individual plasma concentration of zanamivir versus time following intravenous administration in male Sprague-Dawley rats at 1.5 mg/animal from a normal saline (300 μL) formulation (see Example 7). Diamonds refer to test rat #954, squares refer to test rat #955 and triangles refer to test rat #956.

FIG. 23 shows the average plasma concentration of zanamivir versus time following intravenous administration in male Sprague-Dawley rats at 1.5 mg/animal from normal saline (300 μL) formulation (see Example 7).

FIG. 24 shows the individual plasma concentration of zanamivir versus time following intraduodenal (bolus) administration in male Sprague-Dawley rats at 1.5 mg/animal from Capmul MCM L8 (25 μL) formulation (see Example 7). Diamonds refer to test rat #957, squares refer to test rat #958 and triangles refer to test rat #959.

FIG. 25 shows the average plasma concentration of zanamivir versus time following intraduodenal (bolus) administration in male Sprague-Dawley rats at 1.5 mg/animal from Capmul MCM L8 (25 μL) formulation (see Example 7).

FIG. 26 shows the individual plasma concentration of zanamivir versus time following intraduodenal (bolus) administration in male Sprague-Dawley rats at 1.5 mg/animal from Capmul MCM L8 (50 μL) formulation (see Example 7). Diamonds refer to test rat #960, squares refer to test rat #961 and triangles refer to test rat #962.

FIG. 27 shows the average plasma concentration of zanamivir versus time following intraduodenal (bolus) administration in male Sprague-Dawley rats at 1.5 mg/animal from Capmul MCM L8 (50 μL) formulation (see Example 7).

FIG. 28 shows the individual plasma concentration of zanamivir versus time following intraduodenal (bolus) administration in male Sprague-Dawley rats at 1.5 mg/animal from Capmul MCM L8 (75 μL) formulation (see Example 7). Diamonds refer to test rat #963, squares refer to test rat #964 and triangles refer to test rat #965.

FIG. 29 shows the average plasma concentration of zanamivir versus time following intraduodenal (bolus) administration in male Sprague-Dawley rats at 1.5 mg/animal from Capmul MCM L8 (75 μL) formulation (see Example 7).

FIG. 30 shows the average plasma concentration of zanamivir versus time following intraduodenal (bolus) administration in male Sprague-Dawley rats at 1.5 mg/animal from Capmul MCM L8 (25, 50 or 75 μL) formulations (see Example 7). Diamonds refer to Capmul MCM L8@25 μL, squares refer to Capmul MCM L8@50 μL, and triangles refer to Capmul MCM L8@75 μL.

FIG. 31A summarizes the Caco-2 membrane permeability of zanamivir as a function of the vehicle used therefore (i.e., PBS control, glycerol or Capmul MCM L8).

FIG. 31B summarizes the results of additional experiments to determine the Caco-2 membrane permeability of zanamivir as a function of the other vehicle used therefore (i.e., PBS control. 5% glycerol or 0.25% Capmul MCM L8).

FIG. 32A summarizes the absolute bioavailability of 1.5 mg zanamivir administered intraduodenally with 50 μl of various vehicles.

FIGS. 32B and 32C depict results from additional studies using intraduodenal administration of zanamivir/enhancer formulations in male Sprague-Dawley rats. In these experiments, rats fitted with a cannula in the duodenum were administered 1.5 mg of zanamivir in 50 μL vehicles composed of either PBS, glycerol, or Capmul MCM L8. The results demonstrate low absorption of zanamivir in the absence of enhancer, along with dramatically increased absolute bioavailability in their presence. The absolute bioavailability of zanamivir was increased 4.7- and 23.7-fold in 50 μL, of glycerol and Capmul MCM L8, respectively, compared to PBS. In Table 1, the pharmacokinetic parameters for zanamivir using the indicated formulations are presented. Most notably, a Cₘₐₓ of over 7000 ng/mL was achieved when Capmul MCM L8 was used as the enhancer.

As an initial test of the duration of the permeability enhancement effect of glycerol and Capmul MCM L8, experiments were conducted in which the permeability enhancers were administered 2 hr prior to zanamivir dosing. In these experiments, temporal separation of the enhancer and drug by 2 hr resulted in no enhanced absorption; for both enhancers, the absolute bioavailability was equivalent to that of the negative control. Clearly, the enhancement effect is transient and lasts well under 2 hr.

FIG. 33 illustrates the effect of increasing intraduodenally administered Capmul MCM L8 at a fixed 1.5 mg zanamivir drug load on absolute bioavailability.

FIG. 34 summarizes the reciprocal results from varying zanamivir levels at a fixed 50 μL amount of Capmul MCM L8 after intraduodenal administration.

FIG. 35 shows the effect of Capmul MCM L8 on intraduodenal zanamivir absorption in ferrets. Animals (3 per group) were given 10 mg of zanamivir in the indicated vehicle intradoudenally via a surgically placed cannula. The animals were allowed to recover for several days before administration of the test formulation. Blood specimens were obtained at the times indicated, treated with sodium heparin as an anticoagulant, and stored frozen until the levels of zanamivir were quantitated using established LC-MS analytical procedures.

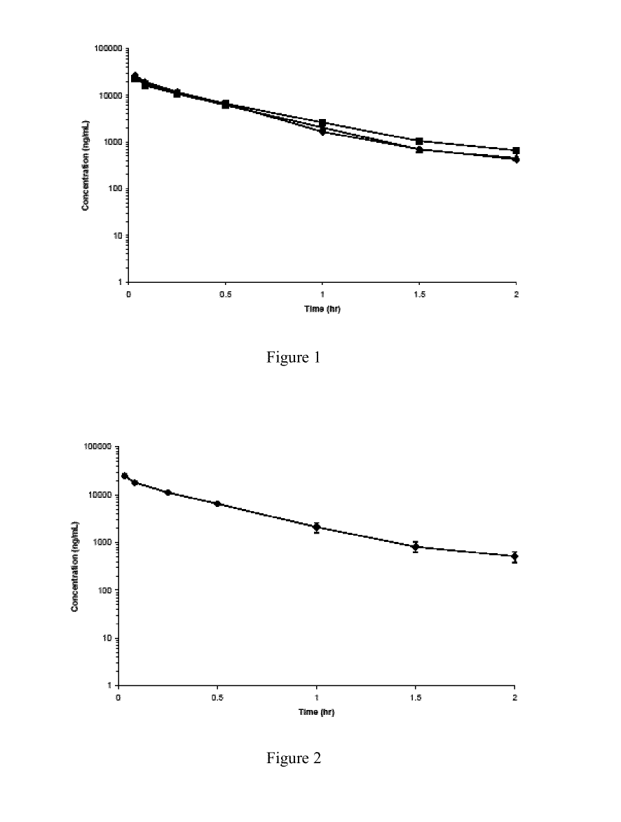

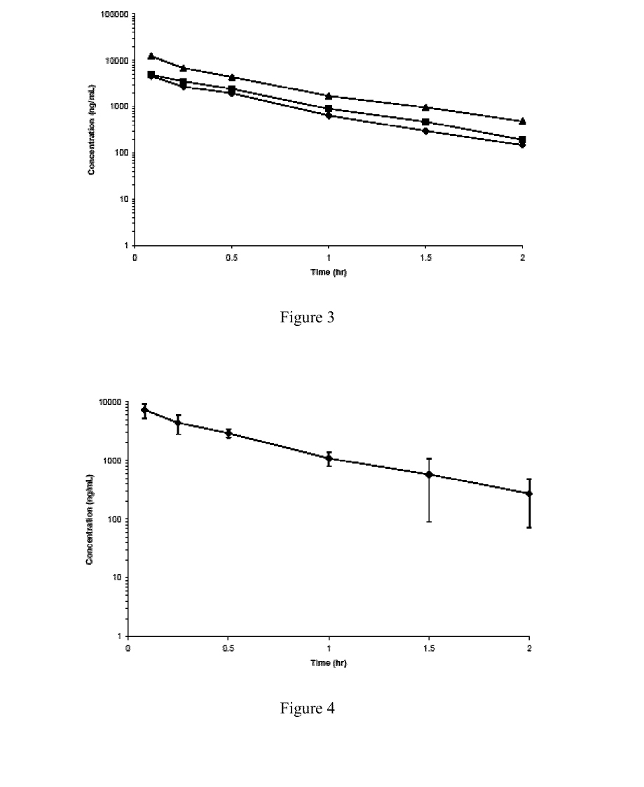

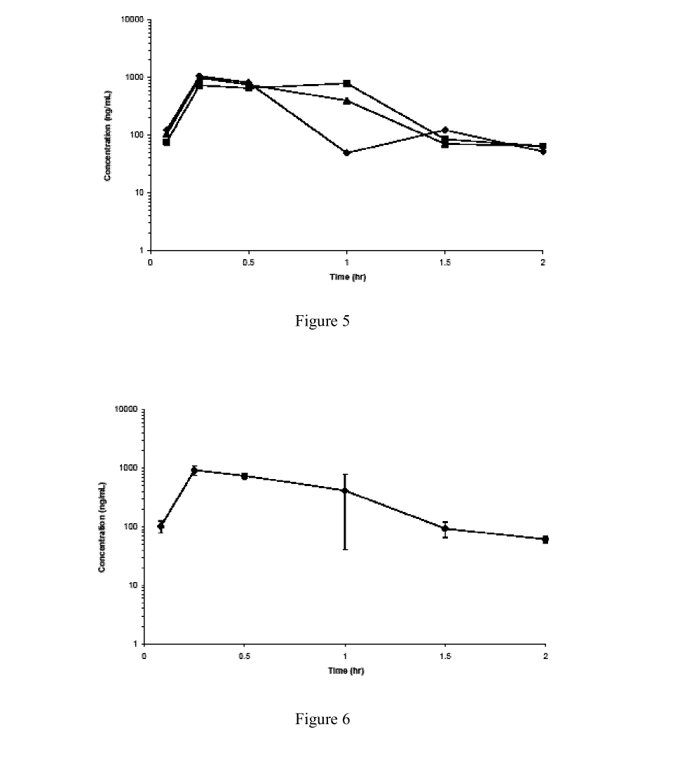

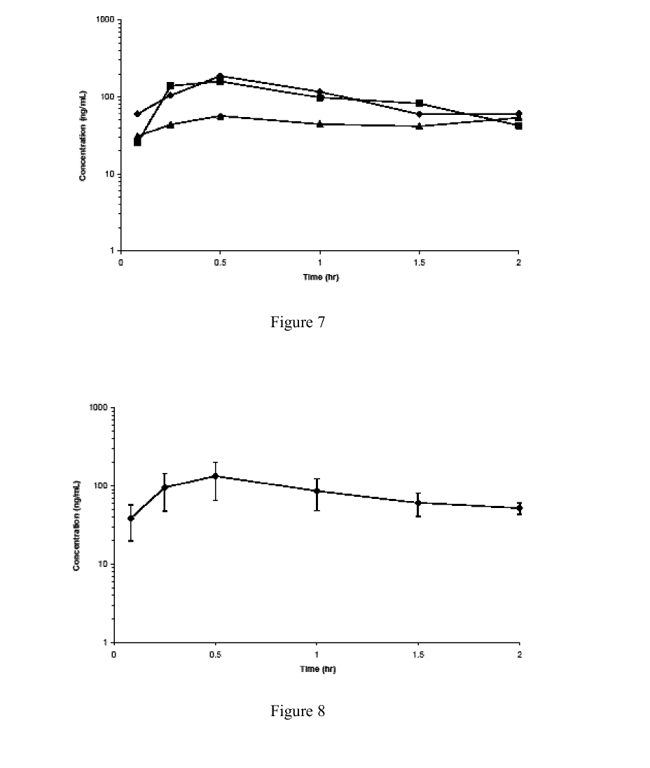

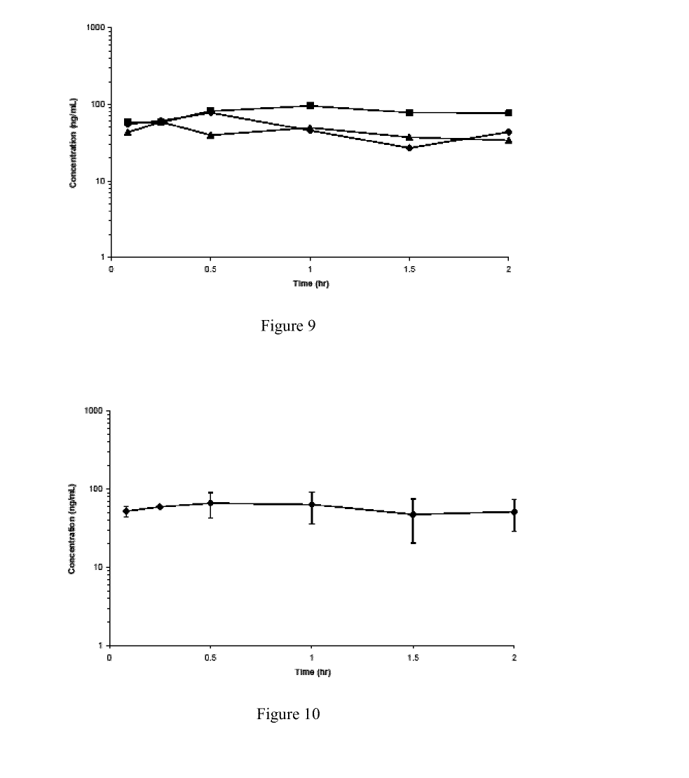

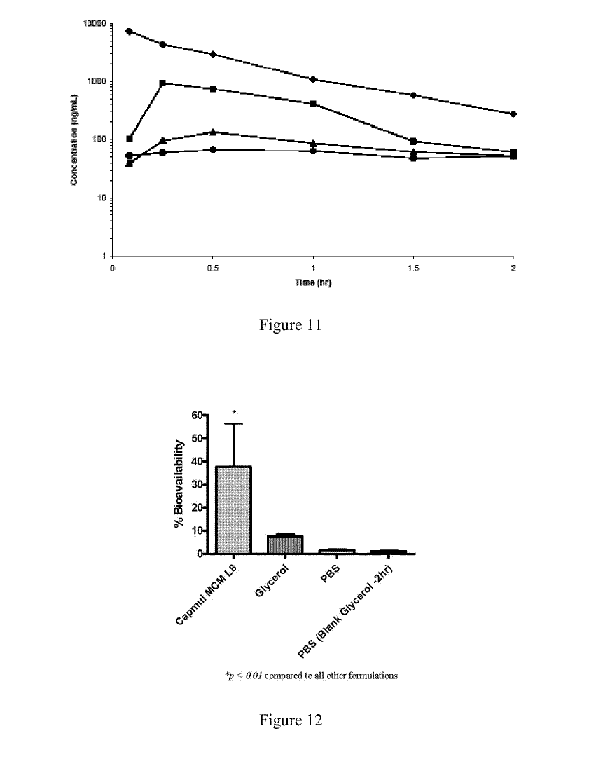

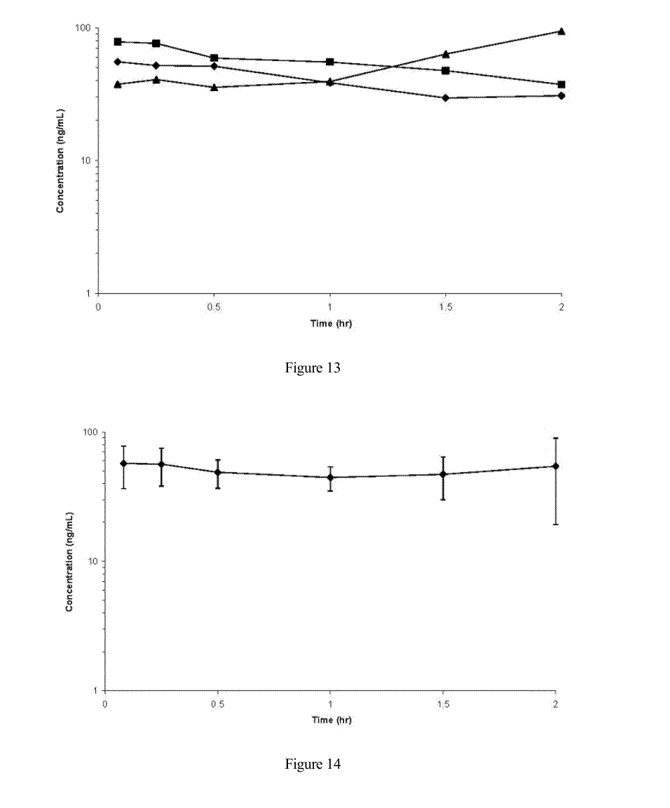

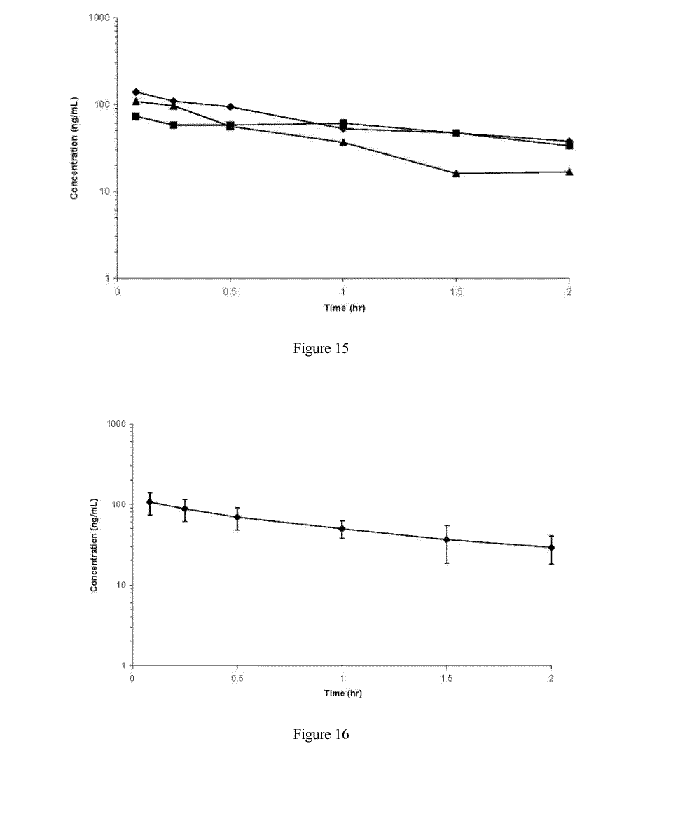

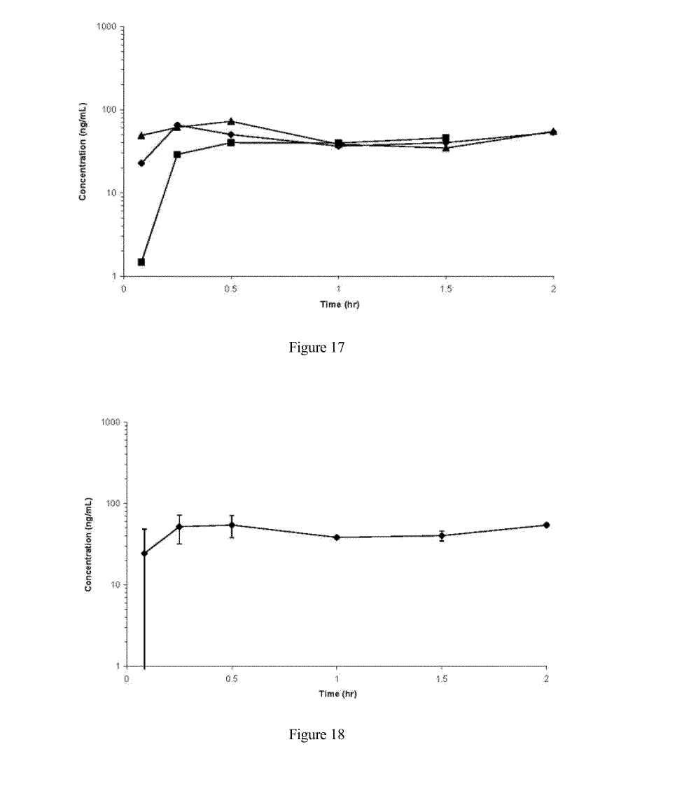

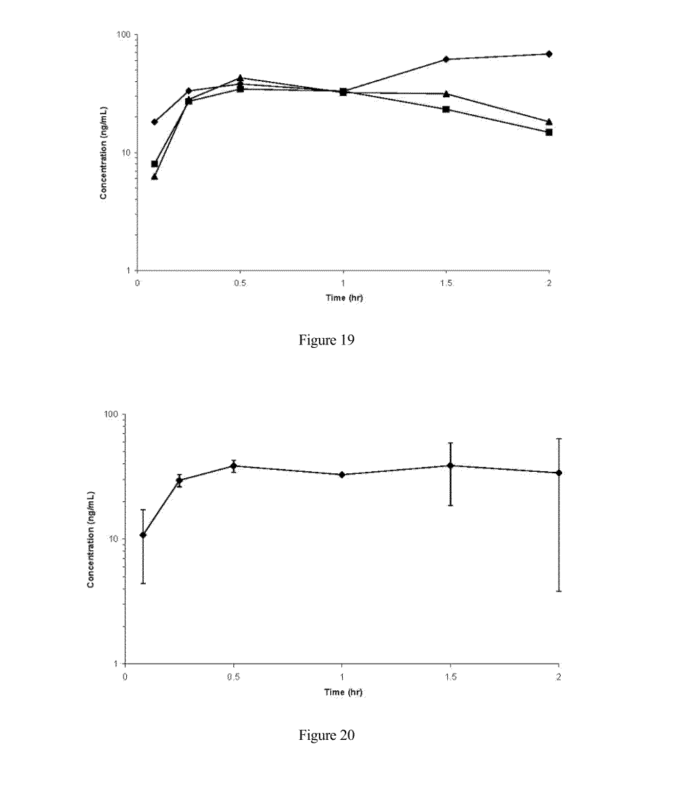

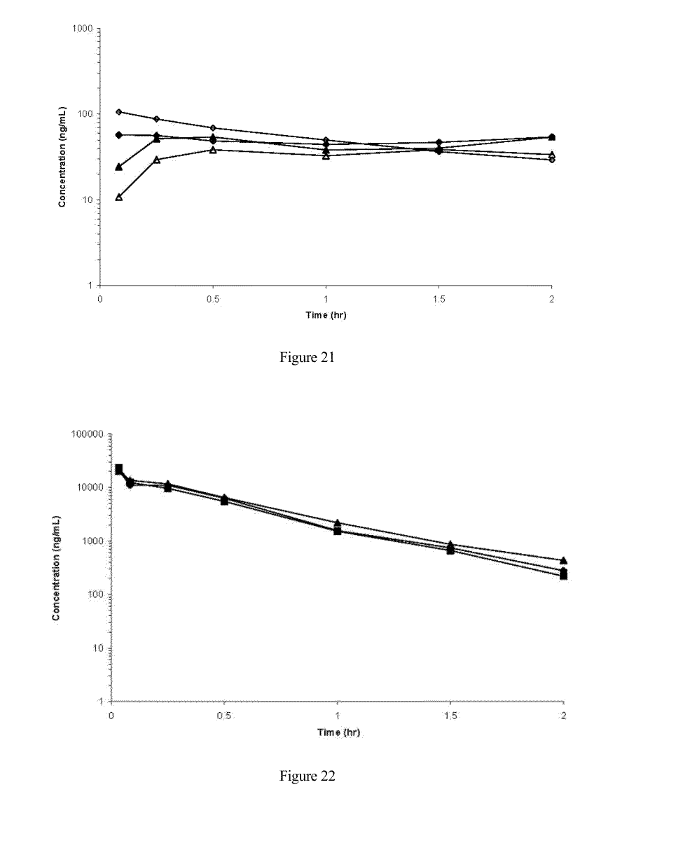

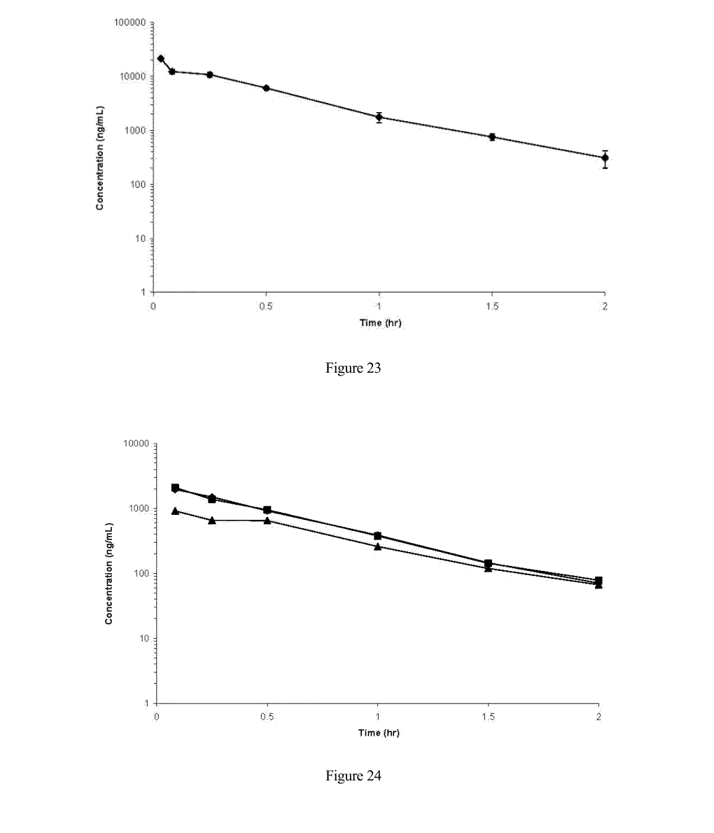

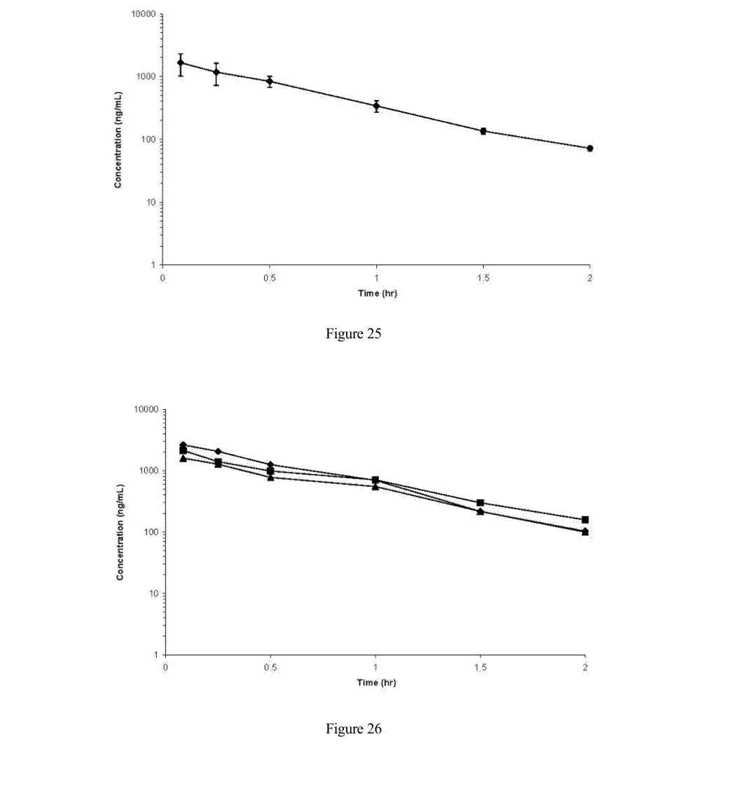

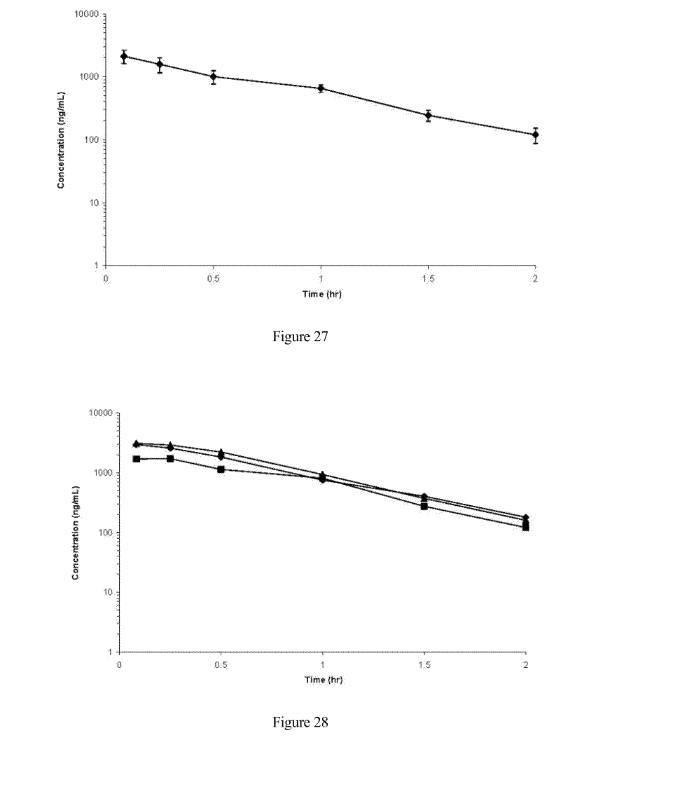

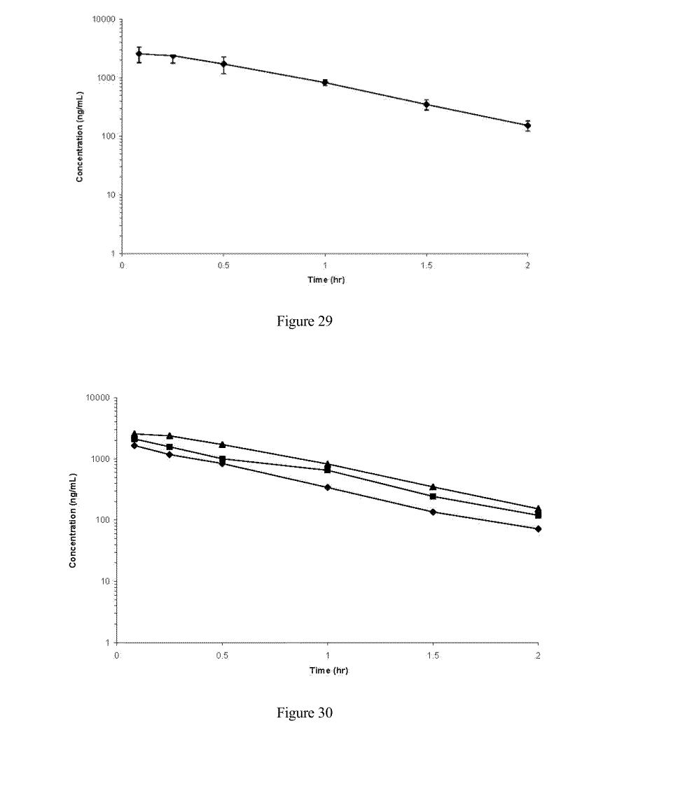

### DETAILED DESCRIPTION OF THE INVENTION

In accordance with the present invention, there are provided compositions comprising:
zanamivir, and
a permeability enhancer,

wherein the composition increases the amount of zanamivir which is transported across the Caco-2 cell membrane by at least 150% relative to the amount of zanamivir which is transported across the Caco-2 cell membrane in the absence of the permeability enhancer.

Zanamivir refers to the compound 5-acetamido-4-guanidino-6-(1,2,3-trihydroxypropyl)-5,6-dihydro-4H-pyran-2-carboxylic acid (zanamivir), and has the chemical structure shown below:

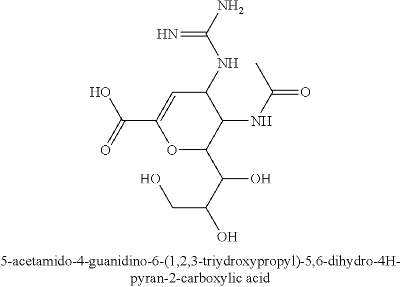 [SMILES: CC(=O)NC1C(NC(=N)N)C=C(C(=O)O)OC1C(O)C(O)CO]

Of particular significance is the presence of three functional groups: an alcohol —OH group, a carboxylic acid group, and a guanidino group. The guanidino group is considered likely to be a major contributor to the improved activity of zanamivir relative to other agents having neuraminidase activity. Yet, the poor oral absorption of zanamivir and alkyl esters thereof may be due in large part to the highly polar nature of the guanidino group, particularly when in the protonated form such as is found in the zwitterionic form of zanamivir. Without intending to be limited to any particular theory or mechanism of action, it is believed that one or more polar groups on the active agent limit permeability of the compound, and this is particularly problematic where the polar active agent is not or is only weakly transported across the cell membrane by a transport protein.

In accordance with the present invention, it has now been found that inclusion of one or more permeability enhancer compounds in formulations with highly polar agents that are poorly absorbed, and in particular, neuraminidase inhibitor formulations (e.g., zanamivir), can increase the amount of active agent that is absorbed by cells, and ultimately increase the bioavailability thereof to the organism. In particular, permeability enhancer compound(s) are believed to provide polar agents such as neuraminidase inhibitors (e.g., zanamivir) with improved oral efficacy with respect to absorption across cellular membranes. Without wishing to be bound by any particular theory or mechanism of action, it is believed that a permeability enhancer compound may facilitate increased absorption of highly polar compounds such as neuraminidase inhibitors (e.g., zanamivir) through cellular tight junctions, may act to promote absorption through a transcellular pathway, or may act to increase permeability through other mechanisms. Accordingly, the invention provides compositions and methods for improving oral bioavailabllity and activity of polar compounds such as neuraminidase inhibitors (e.g., zanamivir).

“Polar” compounds/agents are those that have at least one group that confers a degree of partial or permanent charge on the compound that is greater than or equal to the charge of a hydroxyl group, more preferably greater than or equal to the charge of a carboxyl group, more preferably greater than or equal to the charge of an imidazole group, more preferably greater than or equal to the charge of an amino group, and more preferably greater than or equal to the charge of a guanidino group, phosphate, or sulfate group.

In accordance with another aspect of the present invention, it has been found that variation of either the drug load of compositions according to the present invention, or the amount of enhancer employed therein, demonstrates a generally linear variation in absorption. Thus, compositions according to the present invention are amenable to fine-tuning based on a desired outcome, such as a targeted Cₘₐₓ for enzyme saturation. Conversely, in experiments wherein enhancer was administered 2 hours prior to drug administration, no absorption enhancement was observed. Thus, compositions according to the present invention are unlikely to cause undesirable drug-drug interactions.

Compositions in accordance with the present invention also contemplate oral compositions comprising a therapeutically effective amount of zanamivir and a permeability-enhancing amount of a permeability enhancer. In this aspect, the enhancing amount of permeability enhancer compound is an amount or concentration which produces a zanamivir Caco-2 polar agent permeability at least 150% of (i.e., 1.5-fold over) that provided by zanamivir in the absence of a permeability enhancer.

Compositions in accordance with the present invention also contemplate unit dosage forms comprising a single use dosage of a therapeutically effective amount of zanamivir and a permeability-enhancing amount of a permeability enhancer. In this aspect, the enhancing amount of permeability enhancer compound is an amount or concentration which produces a zanamivir Caco-2 polar agent permeability at least 150% of (i.e., 1.5-fold over) that provided by zanamivir in the absence of a permeability enhancer.

Also contemplated in accordance with the present invention are kits comprising a zanamivir-containing composition as described herein, and directions for the administration thereof to a subject in need thereof.

The invention also provides methods for improving the oral bioavailability of zanamivir, which is not absorbed or only weakly absorbed through a cell membrane. Generally, such methods comprise providing a pharmaceutical formulation comprising a therapeutically effective amount of zanamivir and a permeability-enhancing amount of one or more suitable permeability enhancer compounds in a pharmaceutical formulation or dosage form thereof which is suitable for oral administration. Examples of suitable forms include, for example, capsules, tablets, caplets, various sustained or controlled release dosage forms, solutions, suspensions, and the like, each of which may include acceptable pharmaceutical excipients which are well known to those skilled in the art and suitable for formulation of the dosage form in question.

As used herein, the term “permeability enhancer,” “enhancer” and variations thereof refer to compounds which improve the bioavailability of zanamivir when incorporated into oral formulations. A permeability enhancer may be further defined as a compound capable of increasing the rate of zanamivir transport across a Caco-2 cell membrane by 1.5-fold (150%) or more compared to the zanamivir transport rate in the absence of the enhancer compound. Any means known or otherwise available to those of skill in the art can be used to determine the transport rate, including those Caco-2 cell permeability assays described and exemplified herein.

With respect to the bioavailability of zanamivir, the presence of a permeability enhancer increases the bioavailability of the active agent to the subject relative to the bioavailability of the active agent in the absence of the permeability enhancer. Thus, in some aspects, the presence of the permeability enhancer increases bioavailability of the active agent about 1.5 times the amount of bioavailability of the active agent in the absence of the permeability enhancer. In some embodiments, the presence of the permeability enhancer increases bioavailability of the active agent by about 2 times; in some embodiments, the presence of the permeability enhancer increases bioavailability of the active agent by about 2.5 times; in some embodiments, the presence of the permeability enhancer increases bioavailability of the active agent by about 3 times; in some embodiments, the presence of the permeability enhancer increases bioavailability of the active agent by about 3.5 times; in some embodiments, the presence of the permeability enhancer increases bioavailability of the active agent by about 4 times; in some embodiments, the presence of the permeability enhancer increases bioavailability of the active agent by about 4.5 times; in some embodiments, the presence of the permeability enhancer increases bioavailability of the active agent by about 5 times; in some embodiments, the presence of the permeability enhancer increases bioavailability of the active agent by about 6 times; in some embodiments, the presence of the permeability enhancer increases bioavailability of the active agent by about 7 times; in some embodiments, the presence of the permeability enhancer increases bioavailability of the active agent by about 8 times; in some embodiments, the presence of the permeability enhancer increases bioavailability of the active agent by about 9 times; in some embodiments, the presence of the permeability enhancer increases bioavailability of the active agent by about 10 times; in some embodiments, the presence of the permeability enhancer increases bioavailability of the active agent by about 12 times; in some embodiments, the presence of the permeability enhancer increases bioavailability of the active agent by about 15 times; in some embodiments, the presence of the permeability enhancer increases bioavailability of the active agent by about 17 times; in some embodiments, the presence of the permeability enhancer increases bioavailability of the active agent by about 20 times; in some embodiments, the presence of the permeability enhancer increases bioavailability of the active agent by about 22 times; in some embodiments, the presence of the permeability enhancer increases bioavailability of the active agent by about 25 times; in some embodiments, the presence of the permeability enhancer increases bioavailability of the active agent by about 27 times; in some embodiments, the presence of the permeability enhancer increases bioavailability of the active agent by about 30 times or even greater times the amount of bioavailability of the active agent in the absence of the permeability enhancer.

The invention contemplates that zanamivir, which has low bioavailability in the absence of a permeability enhancer, will have enhanced bioavailability when combined with a permeability enhancer in a formulation. It is desirable that the bioavailability of zanamivir be enhanced by at least about 10% in the subject to which the active agent is administered; in some embodiments, the presence of the permeability enhancer increases bioavailability of the active agent by at least about 15%; in some embodiments, the presence of the permeability enhancer increases bioavailability of the active agent by at least about 20%; in some embodiments, the presence of the permeability enhancer increases bioavailability of the active agent by at least about 25%; in some embodiments, the presence of the permeability enhancer increases bioavailability of the active agent by at least about 30%; in some embodiments, the presence of the permeability enhancer increases bioavailability of the active agent by at least about 35%, more preferably at least about 40%; in some embodiments, the presence of the permeability enhancer increases bioavailability of the active agent by at least about 45%; in some embodiments, the presence of the permeability enhancer increases bioavailability of the active agent by at least about 50%; in some embodiments, the presence of the permeability enhancer increases bioavailability of the active agent by at least about 55%; in some embodiments, the presence of the permeability enhancer increases bioavailability of the active agent by at least about 60%; in some embodiments, the presence of the permeability enhancer increases bioavailability of the active agent by at least about 65%; in some embodiments, the presence of the permeability enhancer increases bioavailability of the active agent by at least about 70%; and in some embodiments, the presence of the permeability enhancer increases bioavailability of the active agent by at least about 75% or more in the subject to which the active agent is administered, when formulated with a permeability enhancer.

Any compound capable of increasing the oral absorption of zanamivir by at least 50% is considered to be within the scope of the present invention. The following examples of permeability enhancers contemplated for use herein are exemplary only and do not constitute a complete list of potential permeability enhancers.

A variety of classes of compounds may serve as suitable permeability enhancers according to the invention. A first category includes fatty acids and salts and esters thereof, including mono-, di-, and triglycerides. Medium chain length fatty acids, especially C8 and C10 acids, and their salts and esters are particularly useful. Suitable specific examples include sodium caprylate, sodium caprate, CAPMUL® glycerides (available from Abitec of Columbus, Ohio), LABRASOL® glycerides (PEG-8 caprylic/capric glycerides, available from Gattefossé SAS of Saint Priest, Cedex, France), GELUCIRE® 44/14 (PEG-32 glyceryl laurate EP, available from Gattefossé), other glycerides & fatty acid esters, CREMOPHOR® (BASF, Ludwigshafen, Germany), D-α-tocopheryl polyethylene glycol 1000 succinate, vegetable oils, polyoxylglycerides, medium chain mono- and diacylglycerides, and the like.

As readily recognized by those of skill in the art, a variety of mixtures of mono- and diglycerides of caprylic and capric acids in glycerol can be employed in the practice of the present invention. For example, mixtures may comprise in the range of 1-99 wt % mono- or diglyceride of caprylic and capric acids (with 5-95 wt % presently preferred), wherein:

the ratio of caprylic acid to capric acid may vary from 1:1 up to 10:1, and

the quantity of free glycerol is preferably no greater than 10 wt %.

One commercially available example of this class, CAPMUL® MCM L8 (glycerol monocaprylate) (available from Abitec of Columbus, Ohio), is composed of mono- and diglycerides of medium chain fatty acids (mainly caprylic, with some capric) and 7% maximum free glycerol. It contains at least 44% alpha monoglycerides (as caprylate).

Other examples of this class of enhancers include GATTEFOSSÉ compositions 61A through 61H which are proprietary to Gattefossé SAS, but generally are composed of mixtures containing one or more of medium chain mono-, di-, or triglycerides, polysorbate derivatives, polyoxyl castor oil derivatives, polyethylene glycol derivatives including polyethylene glycol glycerides, polyoxyl ethers, vegetable oils, and similar GRAS (generally regarded as safe) lipidic components in varying amounts. These components are part of individual commercial products such as CAPRYOL™ 90, CAPRYOL™ PGMC, LAUROGLYCOL™ 90, GELUCIRE® 44/14, Plural Oleique CC497, LABRASOL®, LABRAFIL® M1944CS (apricot kernel oil PEG-6 esters), Transcutol HP, Peceol, and Maisine 35-1, all of which are available from Gattefossé SAS.

While not falling directly within this class, glycerol itself has surprisingly been found to impart excellent permeability enhancement, particularly for neuraminidase inhibitors. This result was not anticipated as glycerol has not previously been considered to be a permeability enhancer.

A second category of enhancers includes surfactants having a steroidal structure, such as bile acid salts. Examples of suitable compounds include sodium cholate, sodium deoxycholate, glycocholate, glycoursodeoxycholate, taurocholate, taurodeoxycholate, and steroid detergents/bile salts. Other surfactants may also be suitable permeability enhancers, including cationic, anionic, and nonionic surfactants.

Examples include polysorbate 80, hexadecyldimethylbenzylammonium chloride, N-hexadecylpyridinium bromide, dodecyltrimethylammonium bromide, hexadecyltrimethylammonium bromide, tetradecyl-8-D-maltoside, octylglucoside, glycyrrhetinic acid, 3-(N,N-dimethylpalmitylammonio)propane-sulfonate, and sodium lauryl sulfate.

Cyclodextrins may also be used as suitable enhancers. Examples include p-cyclodextrin, hydroxypropyl-f3-cyclodextrin, y-cyclodextrin, and hydroxypropyl-y-cyclodextrin.

A variety of other compounds may also be used as enhancers. Examples include sodium salicylate, ethylenediamine tetraacetic acid (EDTA), citric acid, chitosan & chitosan derivatives, N-trimethyl chitosan chloride, monocarboxymethyl-chitosan, palmitoyl carnitine chloride, acyl carnitines, ethylene glycol tetraacetic acid (EGTA), 3-alkylamido-2-alkoxypropyl-phosphocholine derivatives, alkanoylcholines, N-acetylated amino acids (based on a- and non-a-amino acids), mucoadhesive polymers, phospholipids, piperine, 1-methylpiperazine, α-amino acids, and mineral oil.

Thus a wide variety of enhancer compounds may be selected from the group consisting of fatty acids, fatty acid esters, fatty acid salts, glycerol, surfactants, cyclodextrins, sodium salicylate, ethylenedlamine tetraacetic acid, citric acid, chitosan, chitosan derivatives, N-trimethyl chitosan chloride, monocarboxymethyl-chitosan, palmitoyl carnitine chloride, acyl carnitines, ethylene glycol tetraacetic acid, 3-alkylamido-2-alkoxypropyl-phosphocholine derivatives, alkanoylcholines, N-acetylated amino acids, mucoadhesive polymers, phospholipids, piperine, 1-methylpiperazine, α-amino acids, and mineral oil.

The permeability enhancer and zanamivir may be mixed in any proportion so long as there is provided a therapeutically effective amount of zanamivir and a permeability-enhancing amount of the enhancer compound. Enhancement in bioavailability of orally administered zanamivir can depend on the nature and concentration of the enhancer compound with which the zanamivir is formulated. It is thus contemplated that the required therapeutic amount may be contained in a single dosage form or divided between one or more dosages intended for ingestion at the same time or in sequence.

The permeability enhancers act relatively independently of the concentration of polar agent. Differing permeability enhancers can reach either optimal or maximum enhancement over a wide concentration range depending on their particular inherent enhancement potential. Often, enhancers have a non-linear dose response relationship between concentration of enhancer present and amount of increased polar agent absorption. The amount of enhancer to be utilized in an oral dosage form with a polar agent is initially based upon the enhancement properties observed in Caco-2 cell assays at varying fixed enhancer concentrations. Based upon those results, an effective in vivo amount of enhancer compound for a human formulation can be estimated, demonstrated and optimized without undue experimentation using methods well known to those skilled in the formulation art, to achieve a desired pharmacokinetic in vivo profile.

In formulating the composition of this invention, it will be apparent to those skilled in the formulation art that more effective enhancer compounds would require less polar agent than less effective permeability enhancers to achieve a target pharmacokinetic profile.

Given those considerations and variations, in some embodiments, the amount of enhancer may be at least about 0.1 wt % of the combined weight of enhancer and polar agent; in some embodiments, the amount of enhancer may be at least about 1 wt %; in some embodiments, the amount of enhancer may be at least about 10 wt %; in some embodiments, the amount of enhancer may be at least about 20 wt %; in some embodiments, the amount of enhancer may be at least about 30 wt %; in some embodiments, the amount of enhancer may be at least about 40 wt %; in some embodiments, the amount of enhancer may be at least about 50 wt %; in some embodiments, the amount of enhancer may be at least about 60 wt %; in some embodiments, the amount of enhancer may be at least 70 wt % of the combined weight of enhancer and zanamivir. In some embodiments, the amount of enhancer is at most 99 wt %; in some embodiments, the amount of enhancer may be at most 80 wt %; in some embodiments, the amount of enhancer may be at most 75 wt % of the combined weight of the enhancer and polar agent. Thus, as shown in the examples, a typical dosage form may contain a wide range of concentrations of enhancer compounds depending on the compound itself and its efficacy in enhancing the permeability of zanamivir following oral administration. Concentrations as low as 0.001% by weight up to 20% have been demonstrated to be effective in enhancement of the permeability of polar agents (e.g., zanamivir).

Suitable excipients are well known to those skilled in the formulation art, and any excipient or combination of excipients known in the pharmaceutical art may be used. Examples may include flow aids, stabilizers, surface active agents, binders, dispersing agents, flavorings, taste masking agents, coatings, release control agents, water, and/or other excipients typically employed for formulation of oral dosage forms. In some embodiments, the excipient may comprise one or more materials selected from the group consisting of microcrystalline cellulose, dicalcium phosphate, lactose, pre-gelatinized starch, carnauba wax, candelilla wax, silica, and magnesium stearate.

The compositions of this invention may in some aspects be prepared by combining one or more polar agents with suitable amounts of either a single permeability enhancer compound or combinations thereof and optionally with other formulation additives/excipients, mixing thoroughly, and either tableting or filling a suitable hard shell capsule or soft gel capsule with the resulting composition. It has been found that in some cases, sonicating the mixture (i.e., exposure of the neuraminidase inhibitor/enhancer mixture to ultrasonic radiation) may increase the efficacy of the enhancer. Common methods for sonication are known in the art, such as use of a probe or bath sonicator.

It has also been found that in some cases, high-energy blending of the mixture (e.g., exposing the mixture to significant sheer forces) may increase the efficacy of the enhancer. Common methods for high-energy blending include any known in the art, such as stirrers, rotor-stator devices or colloid mills.

It has also been found that in some cases, homogenization or micronization of the mixture (e.g., exposing the mixture to extreme pressure and stress forces, including but not limited to sheer, turbulence, acceleration and impact forces) may increase the efficacy of the enhancer by forming an emulsion of the agent/enhancer mixture in water. Common methods for micronization include any known in the art, such as use of a high pressure homogenizer. Such micronization techniques may significantly reduce the particle size of the mixture in the formulation, providing particle sizes typically <10 μm in size. For example, a CAPMUL® MCM L8/neuraminidase inhibitor mixture may be emulsified in about an equal weight of water. This may be done by repeatedly squirting the mixture through a narrow orifice until an emulsion is formed, or by other emulsion-forming techniques known to those of skill in the art. Although a roughly equal weight of water typically works well, other proportions may also be used according to the invention.

All such methods of sonication, high-energy blending, homogenization and micronization may alter the viscosity of the mixture. It has been found that in some cases, the viscosity of the mixture is significantly increased, sometimes by as much as 50% or more. In some cases, an increase in viscosity may be desirable for improved manufacturability (i.e., improved efficiency of filling solid dosage form vessels such as capsules or soft-gels) or improved content uniformity and decreased variability of the mixture. In some aspects, a significant increase in viscosity may increase the efficacy of the enhancer.

In some aspects, a significant increase in viscosity may indicate a successful endpoint of high-energy mixing, sonication or homogenization. It has also been found that in some cases of homogenization, micronization, sonication or high-energy blending of the mixture, an endothermic reaction may accompany the increase in viscosity. In some embodiments, an endothermic reaction may indicate a successful endpoint of high-energy mixing, sonication or homogenization.

The resulting compositions are typically viscous liquids or paste-like solids. Additional permeability enhancers or formulation additives can either be added prior to sonication or after sonication of the initial lipid/agent composition.

In some embodiments, a tablet, multiparticulate dosage form, capsule, or granule containing the composition may be coated with an enteric or pH-sensitive layer to facilitate drug composition release in the gastro-intestinal tract distal to the stomach. In some embodiments, the enteric coating or pH-sensitive layer may comprise, but is not limited to, one or more materials selected from the group enteric polymers consisting of cellulose acetate phthalate, cellulose acetate trimellitate, hydroxypropyl methylcellulose acetate succinate, hydroxypropyl methylcellulose phthalate, and polyvinyl acetate phthalate; and anionic polymers based on methacrylic acid and methacrylic acid esters.

This disclosure contemplates formulations comprising zanamivir, a permeability enhancer, and optionally other excipients in a tablet or capsule configuration with optional enteric coating. In some embodiments, such compositions are non-aqueous in that water is excluded as a potential excipient and the only water that is present is that which may be present natively or naturally in the individual formulation components. It is also contemplated that the viscosity of liquid formulations for capsule delivery applications according to the invention will be higher than the viscosity of a 5% aqueous solution of that formulation.

In accordance with the present invention, there are also provided methods of treating or preventing influenza infection, said method comprising administering a zanamivir-containing composition as described herein to a subject in need thereof.

In some embodiments, compositions will comprise pharmaceutically acceptable carriers or excipients, such as fillers, binders, disintegrants, glidants, lubricants, complexing agents, solubilizers, surfactants, and the like, which may be chosen to facilitate administration of the compound by a particular route. Examples of carriers include calcium carbonate, calcium phosphate, various sugars such as lactose, glucose, or sucrose, types of starch, cellulose derivatives, gelatin, lipids, liposomes, nanoparticles, and the like. Carriers also include physiologically compatible liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like. Excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil mineral oil, polyethylene glycol (e.g. PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g. 2-hydroxypropyl-.delta.-cyclodextrin), polysorbates (e.g. polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g. a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, hypromellose, and the like.

In some embodiments, oral administration may be used. Pharmaceutical preparations for oral use can be formulated into conventional oral dosage forms such as capsules, tablets, and liquid preparations such as syrups, elixirs, and concentrated drops. Zanamivir and permeability enhancer may be combined with solid excipients, optionally grinding a resulting mixture, and processing the mixture of granules, after adding suitable auxiliaries, if desired, to obtain, for example, tablets, coated tablets, hard capsules, soft capsules, solutions (e.g. aqueous, alcoholic, or oily solutions) and the like. Suitable excipients are, in particular, fillers such as sugars, including lactose, glucose, sucrose, mannitol, or sorbitol; cellulose preparations, for example, corn starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP: povidone); oily excipients, including vegetable and animal oils, such as sunflower oil, olive oil, or codliver oil. The oral dosage formulations may also contain disintegrating agents, such as the cross-linked polyvinylpyrrolidone, agar, or alginic acid, or a salt thereof such as sodium alginate; a lubricant, such as talc or magnesium stearate; a plasticizer, such as glycerol or sorbitol; a sweetening such as sucrose, fructose, lactose, or aspartame; a natural or artificial flavoring agent, such as peppermint, oil of wintergreen, or cherry flavoring; or dye-stuffs or pigments, which may be used for identification or characterization of different doses or combinations. Also provided are dragee cores with suitable coatings. For this purpose, concentrated sugar solutions may be used, which may optionally contain, for example, gum arabic, talc, poly-vinylpyrrolidone, carbopol gel, polyethylene glycol, and/or titanium dioxide, lacquer solutions, and suitable organic solvents or solvent mixtures.

Pharmaceutical preparations that can be used orally include push-fit capsules made of gelatin (“gelcaps”), as well as soft, sealed capsules made of gelatin, and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols.

In some embodiments, injection (parenteral administration) may be used, e.g., intramuscular, intravenous, intraperitoneal, and/or subcutaneous. Zanamivir and permeability enhancing agents for injection may be formulated in sterile liquid solutions, preferably in physiologically compatible buffers or solutions, such as saline solution, Hank's solution, or Ringer's solution. Dispersions may also be prepared in non-aqueous solutions, such as glycerol, propylene glycol, ethanol, liquid polyethylene glycols, triacetin, and vegetable oils. Solutions may also contain a preservative, such as methylparaben, propylparaben, chlorobutanol, phenol, sorbic acid, thimerosal, and the like. In addition, the compositions may be formulated in solid form, including, for example, lyophilized forms, and redissolved or suspended prior to use.

In some embodiments, transmucosal, topical or transdermal administration may be used. In such formulations, penetrants appropriate to the barrier to be permeated are used. Such penetrants are generally known in the art, and include, for example, for transmucosal administration, bile salts and fusidic acid derivatives. In addition, detergents may be used to facilitate permeation. Transmucosal administration, for example, may be through nasal sprays or suppositories (rectal or vaginal). Compositions of compounds of Formula I for topical administration may be formulated as oils, creams, lotions, ointments, and the like by choice of appropriate carriers known in the art. Suitable carriers include vegetable or mineral oils, white petrolatum (white soft paraffin), branched chain fats or oils, animal fats and high molecular weight alcohol (greater than C₁₂). In some embodiments, carriers are selected such that the active ingredient is soluble. Emulsifiers, stabilizers, humectants and antioxidants may also be included as well as agents imparting color or fragrance, if desired. Creams for topical application are preferably formulated from a mixture of mineral oil, self-emulsifying beeswax and water in which mixture the active ingredient, dissolved in a small amount of solvent (e.g., an oil), is admixed. Additionally, administration by transdermal means may comprise a transdermal patch or dressing such as a bandage impregnated with an active ingredient and optionally one or more carriers or diluents known in the art. To be administered in the form of a transdermal delivery system, the dosage administration will be continuous rather than intermittent throughout the dosage regimen.

In some embodiments, compounds are administered as inhalants. Combinations of zanamivir and permeability enhancer may be formulated as dry powder or a suitable solution, suspension, or aerosol. Powders and solutions may be formulated with suitable additives known in the art. For example, powders may include a suitable powder base such as lactose or starch, and solutions may comprise propylene glycol, sterile water, ethanol, sodium chloride and other additives, such as acid, alkali and buffer salts. Such solutions or suspensions may be administered by inhaling via spray, pump, atomizer, or nebulizer, and the like. Combinations of zanamivir and permeability enhancer may also be used in combination with other inhaled therapies, for example corticosteroids such as fluticasone proprionate, beclomethasone dipropionate, triamcinolone acetonide, budesonide, and mometasone furoate; beta agonists such as albuterol, salmeterol, and formoterol; anticholinergic agents such as ipratroprium bromide or tiotropium; vasodilators such as treprostinal and iloprost; enzymes such as DNAase; therapeutic proteins; immunoglobulin antibodies; an oligonucleotide, such as single or double stranded DNA or RNA, siRNA; antibiotics such as tobramycin; muscarinic receptor antagonists; leukotriene antagonists; cytokine antagonists; protease inhibitors; cromolyn sodium; nedocril sodium; and sodium cromoglycate.

The amounts of various compounds to be administered can be determined by standard procedures taking into account factors such as the compound activity (in vitro, e.g. the compound IC₅₀ vs. target, or in vivo activity in animal efficacy models), pharmacokinetic results in animal models (e.g. biological half-life or bioavailability), the age, size, and weight of the subject, and the disorder associated with the subject. The importance of these and other factors are well known to those of ordinary skill in the art. Generally, a dose will be in the range of about 0.01 to 50 mg/kg, also about 0.1 to 20 mg/kg of the subject being treated. Multiple doses may be used.

Combinations of zanamivir and permeability enhancer may also be used in combination with other therapies for treating the same disease. Such combination use includes administration of the invention compositions and one or more other therapeutics at different times, or co-administration of invention compositions and one or more other therapies. In some embodiments, dosage may be modified for invention compositions or other therapeutics used in combination, e.g., reduction in the amount dosed relative to a compound or therapy used alone, by methods well known to those of ordinary skill in the art.

It is understood that use in combination includes use with other therapies, drugs, medical procedures etc., where the other therapy or procedure may be administered at different times (e.g. within a short time, such as within hours (e.g. 1, 2, 3, 4-24 hours), or within a longer time (e.g. 1-2 days, 2-4 days, 4-7 days, 1-4 weeks)) than a composition according to the present invention, or at the same time as an invention composition. Use in combination also includes use with a therapy or medical procedure that is administered once or infrequently, such as surgery, along with a composition according to the invention, administered within a short time or longer time before or after the other therapy or procedure. In some embodiments, the present invention provides for delivery of a composition as described herein and one or more other drug therapeutics delivered by a different route of administration or by the same route of administration. The use in combination for any route of administration includes delivery of an invention composition and one or more other drug therapeutics delivered by the same route of administration together in any formulation, including formulations where the two compounds are chemically linked in such a way that they maintain their therapeutic activity when administered. In one aspect, the other drug therapy may be co-administered with a composition according to the present invention. Use in combination by co-administration includes administration of co-formulations or formulations of chemically joined compounds, or administration of two or more compounds in separate formulations within a short time of each other (e.g. within an hour, 2 hours, 3 hours, up to 24 hours), administered by the same or different routes. Co-administration of separate formulations includes co-administration by delivery via one device, for example the same inhalant device, the same syringe, etc., or administration from separate devices within a short time of each other. Co-formulations including compositions according the present invention, along with one or more additional drug therapies delivered by the same route includes preparation of the materials together such that they can be administered by one device, including the separate compounds combined in one formulation, or compounds that are modified such that they are chemically joined, yet still maintain their biological activity. Such chemically joined compounds may have a linkage that is substantially maintained in vivo, or the linkage may break down in vivo, separating the two active components.

Also provided in accordance with the present invention is the use of zanamivir-containing compositions as described herein in the preparation of a medicament for treating or preventing influenza infection.

The following examples are provided to describe the invention in greater detail. The examples are intended illustrate, not to limit, the invention.

### Example 1

### General Experimental Procedures

Permeability enhancers such as CAPMUL® MCM L8, GATTEFOSSÉ 61A through GATTEFOSSÉ 61H compositions, glycerol, 3-(N,N-dimethylpalmitylammonio)propane sulfonate (PPS), Leuclne, Alanine, Gelucire 44/14, Tween 20, N-methylpiperazine, and d-alpha-tocopheryl polyethylene glycol 1000 succinate (TPGS) were each mixed with zanamivir and vortexed and sonicated. For example, in the case of CAPMUL® MCM L8, the enhancer was mixed with zanamivir in amounts such that the weight ratio of enhancer to zanamivir was in the range of about 333:1 to about 1333:1, and such that when the mixture was subsequently diluted in HBSS to a level at which zanamivir was present at a concentration of 15 mg/mL (0.0015%) the enhancer concentration of the sample was in the range of 0.5% to 2.00%, as shown in the table below. Mixing was conducted by sonication (using either a bath or probe sonicator) which converted the relatively low viscosity liquid mixture to a highly viscous or paste-like composition that is a stable and non-separating.

In like manner, other enhancer compounds were mixed with zanamivir in amounts such that the weight ratio of enhancer to zanamivir was in the range of about 0.7:1 to about 7000:1, and, similarly, such that when the mixture was subsequently diluted to a level at which zanamivir was present at a concentration of 15 pg/mL (0.0015%) the enhancer concentration of the sample was in the range of 0.001% to about 10%, as shown in the table below.

Caco-2 Cell Culture

Caco-2 cells were obtained from American Type Culture Collection (Rockville, Md.). Stock cultures were maintained in flasks with DMEM medium supplemented with 10% FBS, 1% non-essential amino acids, 1 mmol/L sodium pyruvate, 100 IU/mL penicillin, and 100 μg/mL streptomycin in a humidified incubator (37° C., 5% CO₂). Cells were harvested by trypsinization and seeded at 60,000 cells/cm² onto Costar Transwell® 12-well dual-chamber plates with collagen-coated, microporous polycarbonate membranes (1.13 cm² insert area, 0.4 μm pore size; Corning Life Sciences, Acton, Mass.) for permeability studies. The culture medium was changed three times per week.

Certification of Cells for the Study

Caco-2 cell monolayers that had grown for at least 20 days were subjected to batch quality control testing in which permeation rates were measured for atenolol, digoxin, estrone-3-sulfate, lucifer yellow (LY), and propranolol. In addition, the transepithelial electrical resistance (TEER) across each experimental monolayer was tested prior to an experiment.

Tolerability Assessment of Excipients in Caco-2 Cell Monolayers

Tolerability was assessed with zanamivir (15 μg/mL) in the presence and absence of the two excipients (each at 5%) in Caco-2 cell monolayers, based on the rate of permeation of the fluorescent monolayer integrity marker LY immediately post-exposure and after 4 hr recovery.

Unidirectional Permeability of Zanamivir Across Caco-2 Cell Monolayers in the Presence and Absence of Excipients

Unidirectional (A-to-B) permeability of zanamivir was determined at 15 μg/mL in the absence and presence of varying concentrations of excipients in Caco-2 cell monolayers. The assay buffer was Hanks' balanced salt solution (HBSS) supplemented with 10 mM 4-(2-hydroxyethyl)-1-piperazineethanesulfonic acid (HEPES) and 15 mM D-glucose (HBSSg), pH 7.4. The dosing solutions on the apical side (0.5 mL) contained either zanamivir and excipients at each concentration being tested or zanamivir only (n=4), while the receiver buffer in the bottom well (1.5 mL) was always excipient-free. The Caco-2 cell monolayers were incubated for 120 min in a humidified incubator (37° C., 5% CO₂) after dosing in the apical chambers. Aliquots (200 μL each) of receiver buffer were sampled at 60, 90, and 120 min after dosing, with replacement by the same volume of blank buffer (at 60 and 90 min) Donors were sampled at 0 and 120 min.

To assess the viability of the cells after the incubation, the cells were trypsinized and counted, after mixing with trypan blue, with an automated cell counter (Countess™, Invitrogen), which reports total, living, and dead cell numbers, and % viability.

To evaluate the effectiveness of permeability enhancers, data were obtained to demonstrate the ability of one or more permeability enhancer compound(s) to increase zanamivir permeability using Caco-2 cell permeability assays. The assays were performed according to the methods described by Artursson P, Palm K, Luthman K., Caco-2 Monolayers in Experimental and Theoretical Predictions of Drug Transport, Adv Drug Deliv Rev. 2001 Mar. 1; 46(1-3):27-43, and by Shah P, Jogani V, Bagchi T, Misra A., Role of Caco-2 Cell Monolayers in Prediction of Intestinal Drug Absorption, Biotechnol Frog. 2006 January-February; 22(1):186-98. Assays were conducted by seeding approximately 68,000 viable Caco-2 cells in 1.12 cm² Costar Transwell inserts (12-well format, 0.4 micron pore size PET membranes) in Dulbecco's Modified Eagles Medium (high glucose) supplemented with 20% fetal bovine serum, glutamine, pyruvate, non-essential amino acids, epidermal growth factor, ITS (insulin, transferrin, selenium), and penicillin/streptomycin. The cells were incubated for 21-25 days with medium changes every 2-3 days. Transepithelial electrical resistance (TEER) readings were conducted to test the quality of the cell monolayer on the Transwell membrane. The membranes were washed In Hank's Balanced Salt Solution (HBSS, available from Mediatech, Inc., Herndon, Va.) and the resistance across the membrane was measured. Wells having TEER readings of 200 Ωcm² or higher were used in the permeability assays.

Assays were conducted by washing the transwell inserts containing a Caco-2 cell monolayer in HBSS and placing them in 12-well plates with 1.5 ml of HBSS in the lower well. The zanamivir containing test formulation was diluted into HBSS to provide a zanamivir concentration of 15 μg/mL, and 0.5 ml of the solution was added to the transwell insert. Each formulation was tested in triplicate. The transwell inserts were incubated in a 37° C. incubator with rotation at 50 rpm for 30 minutes. At the end of this period the transwell inserts were placed In a fresh 1.5 ml of HBSS in a new well of the 12-well plate and incubated for an additional 30 minutes. A total of 8 to 10 thirty-minute time points were collected by sequentially moving the transwell inserts to fresh 1.5 ml HBSS in successive wells of the 12-well plates. The amount of zanamivir transported into the lower wells was quantitated by LC-MS to define the rate of zanamivir transported across the membrane for each test formulation. The reference control was composed of zanamivir in HBSS in the absence of any permeability enhancer compounds.

As used herein, the term “fold increase” designates the multiplicative effect on zanamivir permeability provided by the enhancer. Thus, the degree of permeability enhancement may be expressed either as a percentage of the permeability of zanamivir alone (in the absence of any permeability enhancing compound or in the presence of a compound which is ineffective in enhancing its permeability), in which case a result of 100% or less indicates no enhancement in permeability. Likewise, these values can be (and are in the figures and in the data sets below) reported as a “fold” value in which 1-fold is equivalent to zanamivir alone (i.e., the same as 100%), a 1.5-fold value is the same as 150% of the value for zanamivir alone, a fold value of 5 is the equivalent of 500% enhancement, and so forth.

Animal Procedures

Male Sprague-Dawley rats (Hilltop Labs), 3 animals per treatment group, 250-350 grams in weight, were fitted with a jugular vein cannula (JVC). Animals intended for intraduodenal dosing were also fitted with one or more intraduodenal cannula (IDC) and those intended for intravenous dosing were fitted with a second JVC. Food was withheld from the animals for a minimum of twelve hours prior to test article administration and was returned approximately four hours post-dose. Water was supplied ad libitum.

Intraduodenal doses composed of 1.5 mg of zanamivir and varying amounts of an enhancer (glycerol or Capmul MCM L8) were injected directly into the duodenum via the IDC. Intravenous dosing was conducted by the injection of 1.5 mg of zanamivir in 200 μL of PBS through a JVC. For some experiments, the absorption enhancer was administered intraduodenally 2 hr prior to administering 1.5 mg of zanamivir in PBS through a second IDC. Each intraduodenal dose was followed with the introduction of a small air bubble (˜10 μL) in the cannula followed by a flush of 125 μL of PBS to ensure the dose was given in full. The volume of PBS used for cannula flush was consistent across the treatment groups. The cannula was tied to prevent the PBS remaining in the cannula from entering the duodenum.

Blood samples collected via the JVC, approximately 400 μL each, were obtained at 2 min, 5 min, 15 min, 30 min, 60 min, 90 min, and 120 min, with sodium heparin used as an anti-coagulant. Each sample was placed into a chilled tube containing the anticoagulant and kept on ice until centrifugation at 4° C., 3,000×g, for 5 min. The plasma supernatants were stored at −70° C. until LC-MS analysis.

### Example 2

### Methodology for Zanamivir

Dosing solution samples were assayed by LC-MS/MS using electrospray ionization. The chromatographic system consisted of Perkin Elmer series 200 micropumps and autosampler equipped with a Waters Atlantic® HILIC Silica 3 μM, 2.1×50 mm column. The mass spectrometer was a PE Sciex API 4000 with electrospray interface in multiple reaction monitoring mode. Specificity of the analytical method was evaluated and neither of the excipients was found to interfere with the analysis of zanamivir. Stock solutions (1 mg/mL zanamivir) were prepared in water. Standards (eight concentrations) were prepared in the appropriate matched matrix (HBSSg or Sprague-Dawley rat plasma containing sodium heparin) and diluted 50-fold with methanol. Experimental samples were treated identically.

Analytical stock solutions (1 mg/mL Zanamivir) were prepared in water.

Standards and samples were prepared in Sprague Dawley rat plasma containing sodium heparin as an anticoagulant. An eight-point calibration curve was prepared at concentrations of 1000, 750, 500, 250, 100, 50, 25, and 10 ng/mL by serial dilution. Standard samples were treated identically to the study samples.

Plasma samples were extracted by protein precipitation in methanol.

| Step | Procedure |
|---|---|
| 1 | Add 20 μL of samples, standards or blanks into 1.5 mL |
| | plastic eppendorf vial. |
| 2 | To each sample, add 980 μL of methanol and vortex for |
| | approximately 30 seconds. |
| 3 | After mixing, centrifuge all samples at approximately |
| | 13,000 rpm for 5 minutes. |
| 4 | Transfer 180 μL of supernatant to 200 μL glass |
| | chromacol HPLC inserts |
| 5 | Cap and inject. |

### HPLC Conditions:

Instrument: Perkin Elmer series 200 micropumps and Autosampler
Column: Waters Atlantic® HILIC Silica 3 μM, 2.1×50 mm column
Aqueous Reservoir (A): 20 mM Ammonium Acetate in water w/1% Methanol
Organic Reservoir (B): 100% Acetonitrile
Gradient Program:

| Time | Gradient | | |
|---|---|---|---|
| (minute) | Curve | % A | % B |
| 0.0 | 1 | 15 | 85 |
| 2.0 | 1 | 15 | 85 |
| 2.1 | 1 | 20 | 80 |
| 3.7 | 1 | 20 | 80 |
| 3.8 | 1 | 40 | 60 |
| 4.3 | 1 | 40 | 60 |
| 4.4 | 1 | 15 | 85 |
| 5.0 | 1 | 15 | 85 |

Flow Rate: 700 μL/min
Injection Volume: 10 μL
Run Time: δ 0 min
Temperature: ambient
Autosampler Wash #1: 1:1:1(v:v:v) water:acetonitrile:isopropanol with 0.2% formic acid
Autosampler Wash#2: 0.1% formic acid in water

### Mass Spectrometer Conditions:

Note: Mass spectrometer conditions may vary between systems and parameters may be optimized as needed.
Instrument: PE Sciex API 4000
Interface: Electrospray (“Turbo Ion Spray”)
Mode: Multiple Reaction Monitoring (MRM)
Gases: CUR 20, CAD 6, GS1 30, GS2 70
Source Temperature: 600° C.
Voltages and Ions Monitored*:

| | | Precursor | Product | | | | | |
|---|---|---|---|---|---|---|---|---|
| Analyte | Polarity | Ion | Ion | IS | DP | EP | CXP | CE |
| Zanamivir | Positive | 333.2 | 60.0 | 3500 | 64 | 9 | 10 | 44 |

IS: Ion Spray Voltage;
DP: Declustering Potential;
EP: Entrance Potential;
CE: Collision Energy;
CXP: Collision Cell Exit Potential;
*All settings are in volts

### Example 3

### Volume of PBS Remaining in the ID Dosing Cannula after the Study

For each intraduodenal dose group, after dosing the 50 μL dosing solution, a flush of a small volume of air pocket and 125 μL of PBS were administered to the dosing cannulae. Approximately 30 μL of the flushing PBS probably entered the duodenum after pushing dosing solution and the air pocket into the duodenum lumen. This was estimated because less resistance was observed from the dosing syringe after the dosing solution and air pocket were administered to the duodenum lumen. After the last sampling time point, the abdominal region of the animal was opened and the intraduodenal cannula was extracted. Air was used to force the remaining PBS through the cannula to be collected into a micro centrifuge tube. Residual PBS droplets adhering to the inside wall of the cannulae were observed. After the best effort to collect the PBS from the cannulae, the volume of the liquid collected from each animal was measured with a pipette, recorded, and subsequently discarded.

Intraduodenal Dose Group of Zanamivir with Capmul MCM L8

| Rat # | 954 | 955 | 956 |
|---|---|---|---|
| Remaining PBS | 20 μL | 36 μL | 40 μL |
| in cannula | | | |

Intraduodenal Dose Group of Zanamivir with Glycerol

| Rat # | 957 | 958 | 959 |
|---|---|---|---|
| Remaining PBS | 29 μL | 28 μL | 30 μL |
| in cannula | | | |

Intraduodenal Dose Group of Zanamivir with PBS

| Rat # | 960 | 961 | 962 |
|---|---|---|---|
| Remaining PBS | 28 μL | 34 μL | 32 μL |
| in cannula | | | |

Intraduodenal Dose Group of Zanamivir with PBS (Blank Glycerol at −2 Hr)—First ID Cannulae was Dosed with Glycerol, Second ID Cannulae Dosed with PBS)

| Rat # | 963 | 964 | 965 |
|---|---|---|---|
| Remaining PBS | 42 μL/45 μL | 31 μL/41 μL | 39 μL/53 μL |
| in cannula | | | |

### Example 4

### Express IV Formulation Development of the Zanamivir (Target: An IV Formulation that could be Prepared in Under One Hour)

The reagents listed in the following table were used in a solubility study.

| Excipient | Supplier |
|---|---|
| Normal Saline | 0.9% aqueous NaCl, in house |

The formulation development studies were performed using the small equipment listed in the table below:

TABLE
Small Equipment

| Equipment/Supply | Supplier | Comments |
|---|---|---|
| Analytical Balance | Mettler-Toledo | AX205 Delta Range ® |
| Printer | Mettler-Toledo | LC-P45 |
| Hotplate/Stirrer | VWR 610 Standard | Serial #040611002 |
| Digital Mini-Vortexer | VWR | Serial #0278 |

The following procedure and potential dosing vehicles were evaluated, and the results are described. The target concentrations of 5 mg/mL were investigated.

Screening Procedure:

1. Weigh 2-5 mg of the zanamivir into a 4 mL glass vial.
2. Add the appropriate volume of normal saline.
3. Vortex, record observations of the formulation.
4. Take an aliquot and dilute it 5-fold with NS.
5. Record observations of the formulation.
6. End of the procedure.

Results

All visual observations are posted below.
Zanamivir was soluble in NS at 5 mg/mL. It passed the 5-fold dilution test with NS. The formulation is recommended for IV dosing in rats.

Formulation Procedure for Zanamivir in Normal Saline at 5 mg/mL:

1. Weigh out required amount of zanamivir powder into a glass vial
2. Add required volume of NS to the powder, vortex to result in a clear solution with 5 mg/mL zanamivir concentration.
3. Dose freshly prepared

### Screening Observations

| | | | | | | | | Obs | |
|---|---|---|---|---|---|---|---|---|---|
| | | | | | | | Suspend- | before | |
| | | Weight | Total | | | Wet | ing | sonication | 5-fold |
| | Conc., | of | volume, | Wet. | Suspending | agent, | agent, | after | dilution |
| Via1#/TC | mg/mil, | TC, mg | mL | Agent, | agent | mL | mL | vortexing | with NS |
| I. Zanamivir | 5.0 | 2.1 | 0.420 | NA | Normal Saline | NA | 0.420 | CS | CS |

### Example 5

### Determination of the Intraduodenal Bioavailability of Zanamivir from Three Different Formulations in Male Sprague-Dawley Rats

Initial screening experiments testing transport of the neuraminidase inhibitor peramivir across Caco-2 cell monolayers, utilizing over 20 potential permeability enhancer compounds or compositions, demonstrated a broad range of impact on drug permeability (results not shown). Two enhancers, glycerol and Capmul MCM L8, provided substantially increased drug transport across Caco-2 cell monolayers and were selected for a more extensive evaluation with an alternate neuraminidase inhibitor, zanamivir, the active ingredient in the drug Relenza® (GSK).

In this example, the bioavailability of Zanamivir was evaluated after intravenous and intraduodenal doses in male Sprague-Dawley rats. The test compound was dosed at 1.5 mg/animal through intravenous and intraduodenal routes from different formulations. Plasma levels were determined by LC-MS/MS analysis. Pharmacokinetic parameters were estimated by a non-compartmental model using WinNonlin v4.1 software.

Following intravenous dosing at 1.5 mg/animal, average Cₘₐₓ values of 30866±3441 ng/mL were observed. The average clearance and volume of distribution were 0.49±0.02 L/hr/kg and 0.24±0.02 L/Kg, respectively. Half life was found to be 0.49±0.03 hours.

After intraduodenal dosing at 1.5 mg/animal from Capmul MCM L8 formulation, Cₘₐₓ of 7233±4390 ng/mL reached at 5 min. Average half-life was 0.49+0.05 hours. The overall percent bioavailability was good, a value of 37.7±18.7.

After intraduodenal dosing at 1.5 mg/animal from glycerol formulation, Cₘₐₓ of 948±136 ng/mL reached between 15 min and 1 hour. The overall percent bioavailability was moderate a value of 7.53±1.07.

After intraduodenal dosing at 1.5 mg/animal from PBS formulation in the absence and presence of intraduodenal blank glycerol predose (50 μl; −2 hr), Cₘₐₓ of 134 68 and 77.4±18.5 ng/mL were observed respectively. The overall percent bioavailability was low, a value of 1.59±0.56 and 1.10±0.39, respectively.

Zanamivir had significantly higher (p<0.01) intraduodenal bioavailability when dosed in Capmul MCM L8 formulation compared to all other formulations. There was no significant difference in bioavailability observed upon intraduodenal pre-dose of blank glycerol.

Dosing Solution Analysis: The dosing solution was analyzed by LC-MS/MS. The measured dosing solution concentration is shown in Table 1. Nominal dosing solution concentrations were used in all calculations. All concentrations are expressed as mg/mL of the free drug.

TABLE 1
Measured Dosing Solution Concentrations (mg/mL)

| | | | | Measured | |
|---|---|---|---|---|---|
| | | | | Dosing | |
| | | | Nominal | Solution | |
| Test | Route of | | Dosing | Concentration | % of |
| Compound | Administration | Vehicle | Conc. (mg/mL) | (mg/mL) | Nominal |
| Zanamivir | IV | Normal Saline | 5.00 | 5.02 | 100 |
| | ID | Capmul MCM | | | |
| | | L8 | 30.0 | 26.1 | 87.1 |
| | | Glycerol | 30.0 | 22.8 | 76.1 |
| | | PBS | 30.0 | 27.7 | 92.4 |

Observations and Adverse Reactions: No adverse reactions were observed after intravenous and intraduodenal dosing of Zanamivir from different formulations in this study.

Sample Analysis: Plasma samples were analyzed using the methods outlined in Appendix I. Plasma concentrations for all compounds are shown in Tables 2-6.

TABLE 2
Individual and Average Plasma Concentrations (ng/mL) and Pharmacokinetic Parameters for
Zanamivir After Intravenous Administration in Male Sprague-Dawley Rats at 1.5 mg/animal
Intravenous (1.5 mg/animal; Normal Saline)

| | Rat # | | | | |
|---|---|---|---|---|---|
| Time (hr) | 951 | 952 | 953 | Mean | SD |
| 0 (predose) | BLOQ | BLOQ | BLOQ | ND | ND |
| 0.033 | 26900 | 22900 | 24600 | 24800 | 2007 |
| 0.083 | 19400 | 17900 | 16300 | 17867 | 1550 |
| 0.25 | 11700 | 11000 | 10800 | 11167 | 473 |
| 0.5 | 6610 | 6630 | 6120 | 6453 | 289 |
| 1.0 | 1650 | 2620 | 2040 | 2103 | 488 |
| 1.5 | 709 | 1050 | 692 | 817 | 202 |
| 2.0 | 423 | 665 | 452 | 513 | 132 |
| Animal Weight (kg) | 0.298 | 0.293 | 0.283 | 0.291 | 0.008 |
| Volume Dosed (mL) | 0.30 | 0.30 | 0.30 | 0.30 | 0.00 |
| Amount Dosed (mg/kg) | 5.03 | 5.12 | 5.30 | 5.15 | 0.14 |
| C₀(ng/mL)¹ | 33376 | 26943 | 32278 | 30866 | 3441 |
| tₘₐₓ (hr)¹ | 0.0 | 0.0 | 0.0 | 0.0 | 0.0 |
| t_{1/2} (hr) | 0.51 | 0.51 | 0.46 | 0.49 | 0.03 |
| CL (L/hr/kg) | 0.49 | 0.47 | 0.52 | 0.49 | 0.02 |
| Vₛₛ (L/kg) | 0.21 | 0.26 | 0.24 | 0.24 | 0.02 |
| AUCₗₐₛₜ (hr · ng/mL) | 9975 | 10118 | 9348 | 9814 | 410 |
| AUCₒₒ (hr · ng/mL) | 10286 | 10603 | 9648 | 10179 | 487 |
| Dose Normalized Values¹ | | | | | |
| AUCₗₐₛₜ (hr · kg · ng/mL/mg) | 1983 | 1976 | 1815 | 1925 | 95.1 |
| AUCₒₒ | 2045 | 2071 | 1873 | 1996 | 107 |

Cₒ: Maximum plasma concentration extrapolated to t = 0;
tₘₐₓ: Time of maximum plasma concentration;
t_{1/2}: half-life, data points used for half-life determination are in bold;
CL: Clearance;
Vₛₛ: Steady state volume of distribution;
AUCₗₐₛₜ: Area Under the Curve, calculated to the last observable time point;
AUCₒₒ: Area Under the Curve, extrapolated to infinity;
ND: Not Determined;
BLOQ: Below the limit of quantitation (10 ng/mL);
¹Extrapolated to t = 0;
²Dose normalized by dividing the parameter by the nominal dose.

TABLE 3
Individual and Average Plasma Concentrations (ng/mL) and Pharmacokinetic Parameters for
Zanamivir After Intraduodenal Administration in Male Sprague-Dawley Rats at 1.5 mg/animal
Intraduodenal (1.5 mg/animal; Capmul MCM L8)

| | Rat # | | | | |
|---|---|---|---|---|---|
| Time (hr) | 954 | 955 | 956 | Mean | SD |
| 0 (pre-dose) | BLOQ | BLOQ | BLOQ | ND | ND |
| 0.83 | 4560 | 4840 | 12300 | 7233 | 4390 |
| 0.25 | 2680 | 3520 | 6850 | 4350 | 2205 |
| 0.5 | 1950 | 2420 | 4370 | 2913 | 1283 |
| 1.0 | 644 | 898 | 1710 | 1084 | 557 |
| 1.5 | 300 | 472 | 962 | 578 | 343 |
| 2.0 | 147 | 193 | 482 | 274 | 182 |
| Animal Weight (kg) | 0.291 | 0.290 | 0.274 | 0.285 | 0.010 |
| Volume Dosed (mL) | 0.05 | 0.05 | 0.05 | 0.05 | 0.00 |
| Amount Dosed (mg/kg) | 5.15 | 5.17 | 5.47 | 5.27 | 0.18 |
| Cₘₐₓ(ng/mL) | 4560 | 4840 | 12300 | 7233 | 4390 |
| Tₘₐₓ (hr) | 0.08 | 0.08 | 0.08 | 0.08 | 0.00 |
| T_{1/2}(hr) | 0.47 | 0.45 | 0.55 | 0.49 | 0.05 |
| AUCₗₐₛₜ (hr · ng/mL) | 2369 | 2980 | 6061 | 3803 | 1979 |
| AUCₒₒ (hr · ng/mL) | 2468 | 3105 | 6442 | 4005 | 2134 |
| Dose Normalized Values¹ | | | | | |
| AUCₗₐₛₜ (hr · kg · ng/mL/mg) | 460 | 576 | 1108 | 715 | 346 |
| AUCₒₒ(hr · kg · ng/mL/mg) | 479 | 601 | 1178 | 753 | 373 |
| Bioavailability (%)² | 24.0 | 30.1 | 59.0 | 37.7 | 18.7 |

Cₐ.: Maximum plasma concentration;
Time of maximum plasma concentration;
T_{1/2}: half-life, data points used for half-life determination are in bold;
AUCₗₐₛₜ: Area Under the Curve, calculated to the last observable time point;
AUCₒₒ: Area Under the Curve, extrapolated to infinity;
ND: Not Determined;
BLOQ: Below the limit of quantitation (10 ng/mL);
¹Dose normalized by dividing the parameter by the nominal dose;
²Bioavailability determined by dividing the individual dose normalized intraduodenal AUCₒₒ values by the average dose normalized IV AUCₒₒ value.

TABLE 4
Individual and Average Plasma Concentrations (ng/mL) and
Pharmacokinetic Parameters for Zanamivir After Intraduodenal
Administration in Male Sprague-Dawley Rats at 1.5 mg/animal
Intraduodenal (1.5 mg/animal; Glycerol)

| | Rat # | | | | |
|---|---|---|---|---|---|
| Time (hr) | 957 | 958 | 959 | Mean | SD |
| 0 (pre-dose) | BLOQ | BLOQ | BLOQ | ND | ND |
| 0.83 | 123 | 77.1 | 105 | 102 | 23.1 |
| 0.25 | 1060 | 737 | 986 | 928 | 169 |
| 0.5 | 817 | 659 | 755 | 744 | 79.6 |
| 1.0 | 49.2 | 797 | 400 | 415 | 374 |
| 1.5 | 123 | 85.1 | 71 | 93.0 | 26.9 |
| 2.0 | 52.1 | 65.3 | 65.6 | 61.0 | 7.71 |
| Animal Weight (kg) | 0.288 | 0.284 | 0.293 | 0.288 | 0.005 |
| Volume Dosed (mL) | 0.05 | 0.05 | 0.05 | 0.05 | 0.00 |
| Amount Dosed (mg/kg) | 5.21 | 5.28 | 5.12 | 5.20 | 0.08 |
| Cₘₐₓ (ng/mL) | 1060 | 797 | 986 | 948 | 136 |
| tₘₐₓ (hr) | 0.25 | 1.00 | 0.25 | 0.50 | 0.43 |
| t_{1/2} (hr) | ND¹ | ND² | 0.29 | 0.29 | ND |
| AUCₗₐₛₜ (hr · ng/mL) | 642 | 868 | 754 | 754 | 113 |
| AUCₒₒ (hr · ng/mL) | ND¹ | ND² | 781 | 781 | ND |
| Dose Normalized Values³ | | | | | |
| AUCₗₐₛₜ | 123 | 164 | 147 | 145 | 20.7 |
| (hr · kg · ng/mL/mg) | | | | | |
| AUCₒₒ | ND¹ | ND² | 153 | 153 | ND, |
| (hr · kg · ng/mL/mg) | | | | | |
| Bioavailability (%)⁴ | 6.04 | 8.54 | 7.65 | 7.53 | 1.07 |

Cₘₐₓ: Maximum plasma concentration;
tₘₐₓ: Time of maximum plasma concentration;
t_{1/2}: half-life, data points used for half-life determination are in bold;
AUCₗₐₛₜ: Area Under the Curve, calculated to the last observable time point;
AUCₒₒ: Area Under the Curve, extrapolated to infinity;
ND: Not Determined;
BLOQ: Below the limit of quantitation (10 ng/mL);
¹not determined due to lack of quantifiable data points trailing the Cₘₐₓ;
²not determined due to correlation coefficient (R²) was less than 0.85;
³Dose normalized by dividing the parameter by the nominal dose;
Bioavailability determined by dividing the individual dose normalized intraduodenal AUCₗₐₛₜ values by the average dose normalized IV AUCₗₐₛₜ value.

TABLE 5
Individual and Average Plasma Concentrations (ng/mL) and
Pharmacokinetic Parameters After Intraduodenal Administration in
Male Sprague-Dawley Rats at 1.5 mg/animal
Intraduodenal (1.5 mg/animal; PBS)

| | Rat # | | | | |
|---|---|---|---|---|---|
| Time (hr) | 960 | 961 | 962 | Mean | SD |
| 0 (pre-dose) | BLOQ | BLOQ | BLOQ | ND | ND |
| 0.83 | 60.2 | 25.2 | 30.7 | 38.7 | 18.8 |
| 0.25 | 105 | 139 | 43.9 | 96.0 | 48.2 |
| 0.5 | 186 | 159 | 56.4 | 134 | 68.4 |
| 1.0 | 117 | 97.9 | 44.2 | 86.4 | 37.7 |
| 1.5 | 59.6 | 82 | 41.6 | 61.1 | 20.2 |
| 2.0 | 60.2 | 42.6 | 53.4 | 52.1 | 8.88 |
| Animal Weight (kg) | 0.274 | 0.287 | 0.300 | 0.287 | 0.013 |
| Volume Dosed (mL) | 0.05 | 0.05 | 0.05 | 0.05 | 0.00 |
| Amount Dosed (mg/kg) | 5.47 | 5.23 | 5.00 | 5.23 | 0.24 |
| Cₘₐₓ (ng/mL) | 186 | 159 | 56 | 134 | 68 |
| tₘₐₓ (hr) | 0.50 | 0.50 | 0.50 | 0.50 | 0.00 |
| t_{1/2} (hr) | ND¹ | 0.83 | ND² | 0.83 | ND |
| AUCₗₐₛₜ (hr · ng/mL) | 203 | 192 | 90 | 162 | 62.0 |
| AUCₒₒ (hr · ng/mL) | ND¹ | 244 | ND² | 244 | ND |
| Dose Normalized Values³ | | | | | |
| AUCₗₐₛₜ (hr · kg · ng/mL/mg) | 37.0 | 36.8 | 18.1 | 30.6 | 10.9 |
| AUCₒₒ (hr · kg · ng/mL/mg) | ND¹ | 46.6 | ND² | 46.6 | ND |
| Bioavailability (%)⁴ | 1.92 | 1.91 | 0.94 | 1.59 | 0.56 |

Cₘₐₓ: Maximum plasma concentration;
tₘₐₓ: Time of maximum plasma concentration;
t_{1/2}: half-life, data points used for half-life determination are in bold;
AUCₗₐₛₜ: Area Under the Curve, calculated to the last observable time point;
AUCₒₒ: Area Under the Curve, extrapolated to infinity;
ND: Not Determined;
BLOQ: Below the limit of quantitation (10 ng/mL);
¹not determined due to correlation coefficient (R²) was less than 0.85;
²not determined due to lack of quantifiable data points trailing the Cₘₐₓ;
³Dose normalized by dividing the parameter by the nominal dose;
⁴Bioavailability determined by dividing the individual dose normalized intraduodenal AUCₗₐₛₜ values by the average dose normalized IV AUCₗₐₛₜ value.

TABLE 6
Individual and Average Plasma Concentrations (ng/mL) and
Pharmacokinetic Parameters for Zanamivir After Intraduodenal
Administration in Male Sprague-Dawley Rats at 1.5 mg/animal
Intraduodenal (1.5 mg/animal; PBS, 50 pL Blank Glycerol-2 hr)

| | Rat # | | | | |
|---|---|---|---|---|---|
| Time (hr) | 963 | 964 | 965 | Mean | SD |
| 0 (pre-dose) | BLOQ | BLOQ | BLOQ | ND | ND |
| 0.83 | 55.3 | 58.1 | 43.2 | 52.2 | 7.92 |
| 0.25 | 60.9 | 58.1 | 58.9 | 59.3 | 1.44 |
| 0.5 | 77.5 | 81.8 | 39.6 | 66.3 | 23.2 |
| 1.0 | 45.7 | 95.8 | 49.5 | 63.7 | 27.9 |
| 1.5 | 26.9 | 78.2 | 37.3 | 47.5 | 27.1 |
| 2.0 | 43.7 | 76.7 | 33.9 | 51.4 | 22.4 |
| Animal Weight (kg) | 0.276 | 0.284 | 0.284 | 0.281 | 0.005 |
| Volume Dosed (mL) | 0.05 | 0.05 | 0.05 | 0.05 | 0.00 |
| Amount Dosed (mg/kg) | 5.43 | 5.28 | 5.28 | 5.33 | 0.09 |
| Cₘₐₓ (ng/mL) | 77.5 | 95.8 | 58.9 | 77.4 | 18.5 |
| tₘₐₓ (hr) | 0.50 | 1.00 | 0.25 | 0.58 | 0.38 |
| t_{1/2} (hr) | 0.66 | ND¹ | 1.83 | 1.24 | ND |
| AUCₗₐₛₜ (hr · ng/mL) | 95.9 | 156 | 84.4 | 112 | 38.6 |
| AUCₒₒ (hr · ng/mL)² | 137 | ND¹ | 174 | 156 | ND |
| Dose Normalized Values³ | | | | | |
| AUCₗₐₛₜ (hr · kg · ng/mL/mg) | 17.7 | 29.6 | 16.0 | 21.1 | 7.42 |
| AUCₒₒ (hr · kg · ng/mL/mg) | 25.3 | ND¹ | 32.9 | 29.1 | ND |
| Bioavailability (%)⁴ | 0.92 | 1.54 | 0.83 | 1.10 | 0.39 |

Cₘₐₓ: Maximum plasma concentration;
tₘₐₓ: Time of maximum plasma concentration;
t_{1/2}: half-life, data points used for half-life determination are in bold;
AUCₗₐₛₜ: Area Under the Curve, calculated to the last observable time point;
AUC.: Area Under the Curve, extrapolated to infinity;
ND: Not Determined; Below the limit of quantitation (10 ng/mL);
¹not determined due to correlation coefficient (R²) was less than 0.85;
²AUCₒₒ, is a greater than 25% extrapolation above its respective AUCₗₐₛₜ value;
³Dose normalized by dividing the parameter by the nominal dose;
⁴Bioavailability determined by dividing the individual dose normalized intraduodenal AUCₗₐₛₜ values by the average dose normalized IV AUCₗₐₛₜ value.

Summary of pharmacokinetic results are presented in Table 7.

TABLE 7
Summary of Pharmacokinetic Parameters for Zanamivir From
Different Formulations after Intraduodenal Administration in
Male Sprague-Dawley Rats at 1.5 mg/animal

| | Intraduodenal | | | |
|---|---|---|---|---|
| | | | | PBS |
| | | | | (Blank |
| | Capmul | | | Glycerol- |
| PK parameter | MCM L8 | Glycerol | PBS | 2 hr) |
| C_{maz} (ng/mL) | 7233 | 948 | 134 | 77.4 |
| tₘₐₓ (hr) | 0.08 | 0.50 | 0.50 | 0.58 |
| t_{1/2} (hr) | 0.49 | 0.29 | 0.83 | 1.24 |
| AUCₗₐₛₜ (hr · ng/mL) | 3803 | 754 | 162 | 112 |
| AUCₒₒ (hr · ng/mL) | 4005 | 781 | 244 | 156 |
| Dose Normalized Values¹ | | | | |
| AUCₗₐₛₜ (hr · kg · ng/mL/mg) | 715 | 145 | 30.6 | 21.1 |
| AUCₒₒ (hr · kg · ng/mL/mg) | 753 | 153 | 46.6 | 29.1 |
| Bioavailability (%) | 37.7 | 7.53 | 1.59 | 1.10 |

Cₘₐₓ: Maximum plasma concentration;
tₘₐₓ: Time of maximum plasma concentration;
t_{1/2}: half-life,
AUCₗₐₛₜ: Area Under the Curve, calculated to the last observable time point
AUCₒₒ: Area Under the Curve, extrapolated to infinity;
¹Dose normalized by dividing the parameter by the nominal dose of 1.5 mg/animal.

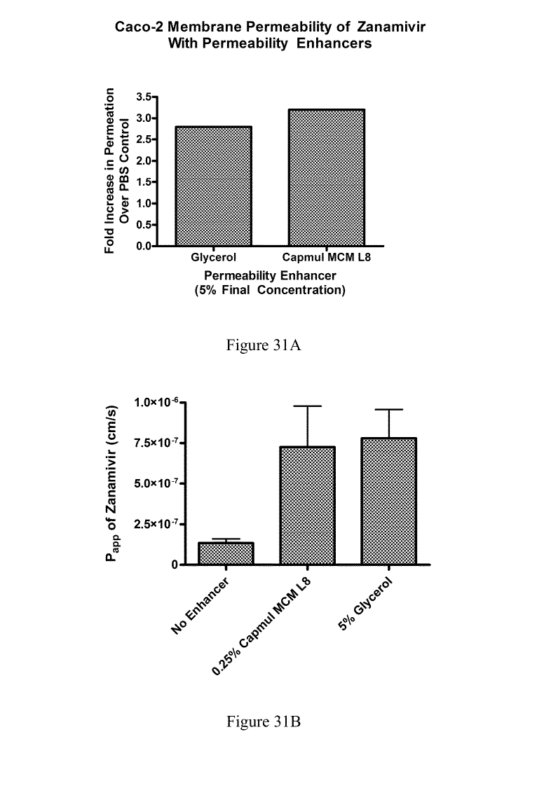

FIG. 31B shows results of Caco-2 cell assays with zanamivir utilizing optimized concentrations of both Capmul MCM L8 and glycerol. Optimized concentrations of each were derived based upon conditions found to provide the highest permeability and no impact on Caco-2 cell viability and membrane integrity (viability and tolerability test results not shown). Under the optimized conditions, both Capmul MCM L8 and glycerol provided over a 5-fold increase in the apparent permeability coefficient (Pₐₚₚ) of zanamivir. Capmul MCM L8 was an inherently more potent permeability enhancer with zanamivir, as a similar increase in zanamivir transport across the membrane was observed at a 20-fold lower concentration than with glycerol. These results demonstrated a clear ability for permeability enhancers to increase the transport of zanamivir across a biological barrier (a monolayer of intestinal epithelial cells); therefore, the study was expanded to explore the potential of these enhancers for increased intestinal absorption in a rat model system.

### Impact of Permeability Enhancers on Zanamivir Absolute Bioavailability in Rats.

Results from Caco-2 cell monolayer permeability studies suggested the permeability enhancers glycerol and Capmul MCM L8 could provide a significant increase in zanamivir absorption despite its high polarity and inherently low absolute bioavailability of under 2%.

FIG. 31C depicts results from studies using intraduodenal administration of zanamivir/enhancer formulations in male Sprague-Dawley rats. In these experiments, rats fitted with a cannula in the duodenum were administered 1.5 mg of zanamivir in 50 μL vehicles composed of either PBS, glycerol, or Capmul MCM L8. The results demonstrate low absorption of zanamivir in the absence of enhancer, along with dramatically increased absolute bioavailability in their presence. The absolute bioavailability of zanamivir was increased 4.7- and 23.7-fold in 50 μL of glycerol and Capmul MCM L8, respectively, compared to PBS. In Table 1, the pharmacokinetic parameters for zanamivir using the indicated formulations are presented. Most notably, a Cₘₐₓ of over 7000 ng/mL was achieved when Capmul MCM L8 was used as the enhancer.

As an initial test of the duration of the permeability enhancement effect of glycerol and Capmul MCM L8, experiments were conducted in which the permeability enhancers were administered 2 hr prior to zanamivir dosing. In these experiments, temporal separation of the enhancer and drug by 2 hr resulted in no enhanced absorption; for both enhancers, the absolute bioavailability was equivalent to that of the negative control. Clearly, the enhancement effect is transient and lasts well under 2 hr.

### Example 6

### Determination of the Intraduodenal Bioavailability of Zanamivir from Different Formulations in Male Sprague-Dawley Rats

In this example, the bioavailability of Zanamivir was evaluated after intraduodenal doses in male Sprague-Dawley rats. The test compound was dosed at 1.5 mg/animal through intraduodenal routes from glycerol and PBS formulations. Plasma levels were determined by LC-MS/MS analysis. Pharmacokinetic parameters were estimated by a non-compartmental model using WinNonlin v5.2.1 software.

After intraduodenal dosing at 1.5 mg/animal from the glycerol (100 μL) formulation, Cₘₐₓ of 76.1±19.7 ng/mL was reached between 5 min and 2 hours. The single determined half-life was 1.77 hours. The overall percent bioavailability was low with a value of 0.97±0.17.

After intraduodenal dosing at 1.5 mg/animal from the glycerol (150 μL) formulation, Cₘₐₓ of 107+33.2 ng/mL was reached at 5 min. The average half-life was 1.63 hours (n=2). The overall percent bioavailability was low with a value of 1.09±0.23.

After intraduodenal dosing at 1.5 mg/animal from the PBS formulation in presence of the intraduodenal blank glycerol (150 μL−2 hr) predose, Cₘₐₓ of 61.1±13.7 ng/mL was observed between 15 min and 1.5 hours. The overall percent bioavailability was low with a value of 0.79±0.25.

After intraduodenal dosing at 1.5 mg/animal from PBS formulation in presence of the intraduodenal blank Capmul MCM L8 (50 μL; −2 hr) predose, Cₘₐₓ of 48.6±17.6 ng/mL was observed between 30 min and 2.0 hours. The single determined half-life was 0.86 hours. The overall percent bioavailability was low with a value of 0.66±0.21.

With increase in the glycerol dose volume from 50 to 100 and 1504 L, the ID bioavailability decreased significantly. There was a decrease in bioavailability observed upon intraduodenal pre-treatment with blank glycerol (50 μL and 150 μL) and blank Capmul MCM L8 (50 μL) compared to the untreated PBS dosed group.

The dosing solution was analyzed by LC-MS/MS. The measured dosing solution concentrations are shown in Table 8. Nominal dosing solution concentrations were used in all calculations. All concentrations are expressed as mg/mL of the free drug.

TABLE 8
Measured Dosing Solution Concentrations mg/mL

| | | | | Measured | |
|---|---|---|---|---|---|
| | | | | Dosing | |
| | | | Nominal | Solution | |
| | Route of | | Dosing | Concen- | |
| Test | Adminis- | | Conc. | tration | % of |
| Compound | tration | Vehicle | (mg/mL) | (mg/mL) | Nominal |
| Zanamivir | ID | Glycerol | 15.0 | 13.9 | 92.7 |
| | | | 10.0 | 8.80 | 88.0 |
| | | PBS | 30.0 | 25.7 | 85.7 |

Plasma samples were analyzed using the methods outlined in Examples 2, 3 and 4. Plasma concentrations for Zanamivir are shown in Tables 9-12.

TABLE 9
Individual and Average Plasma Concentrations (ng/mL) and
Pharmacokinetic Parameters for Zanamivir After Intraduodenal
Administration in Male Sprague-Dawley Rats at 1.5 mg/animal
Intraduodenal (1.5 mg/animal; 100•μL Glycerol

| | Rat # | | | | |
|---|---|---|---|---|---|
| Time (hr) | 181 | 182 | 183 | Mean | SD |
| 0 (pre-dose) | BLOQ | BLOQ | BLOQ | ND | ND |
| 0.83 | 55.4 | 78.4 | 37.5 | 57.1 | 20.5 |
| 0.25 | 52.0 | 76.4 | 40.7 | 56.4 | 18.2 |
| 0.5 | 51.4 | 59.2 | 35.6 | 48.7 | 12.0 |
| 1.0 | 38.5 | 55.3 | 39.4 | 44.4 | 9.5 |
| 1.5 | 29.6 | 47.6 | 63.5 | 46.9 | 17.0 |
| 2.0 | 30.8 | 37.4 | 94.5 | 54.2 | 35.0 |
| Animal Weight (kg) | 0.291 | 0.299 | 0.280 | 0.290 | 0.010 |
| Volume Dosed (mL) | 0.100 | 0.100 | 0.100 | 0.100 | 0.000 |
| Amount Dosed (mg/kg) | 5.15 | 5.02 | 5.36 | 5.18 | 0.17 |
| Cₘₐₓ (ng/mL) | 55.4 | 78.4 | 94.5 | 76.1 | 19.7 |
| tₘₐₓ (hr) | 0.08 | 0.08 | 2.00 | 0.72 | 1.11 |
| t_{1/2} (hr) | ND¹ | 1.77 | ND′ | 1.77 | ND |
| AUCₗₐₛₜ (hr · ng/mL) | 78.8 | 109 | 102 | 96.4 | 15.6 |
| AUCₒₒ (hr · ng/mL) | ND¹ | 204² | ND¹ | 204 | ND |
| Dose Normalized Values³ | | | | | |
| AUCₗₐₛₜ | 15.3 | 21.7 | 19.0 | 18.6 | 3.19 |
| (hr · kg · ng/mL/mg) | | | | | |
| AUCₒₒ | ND¹ | 40.7² | ND¹ | 40.7 | ND |
| (hr · kg · ng/mL/mg) | | | | | |
| Bioavailability (%)⁴ | 0.79 | 1.13 | 0.98 | 0.97 | 0.17 |

Cₘₐₓ: Maximum plasma concentration;
tₘₐₓ: Time of maximum plasma concentration;
t_{1/2}: half-life, data points used for half-life determination are in bold;
AUCₗₐₛₜ: Area Under the Curve, calculated to the last observable time point;
AUCₒₒ: Area Under the Curve, extrapolated to infinity;
ND: Not Determined;
BLOQ: Below the limit of quantitation (10 ng/mL);
′not determined due to terminal log linear phase not observed;
²AUC,, is a greater than 25% extrapolation above its respective AUCₗₐₛₜ value;
³Dose normalized by dividing the parameter by the actual dose;
⁴Bioavailability determined by dividing the individual dose normalized intraduodenal AUCₗₐₛₜ values by the average dose normalized IV AUCₗₐₛₜ value from 11HAWAP1R1.

TABLE 10
Individual and Average Plasma Concentrations (ng/mL) and
Pharmacokinetic Parameters for Zanamivir After Intraduodenal
Administration in Male Sprague-Dawley Rats at 1.5 mg/animal
Intraduodenal (1.5 m animal; 150 μL Glycerol)

| | Rat # | | | | |
|---|---|---|---|---|---|
| Time (hr) | 184 | 185 | 186 | Mean | SD |
| 0 (pre dose) | BLOQ | BLOQ | BLOQ | ND | ND |
| 0.83 | 139 | 72.7 | 108 | 107 | 33.2 |
| 0.25 | 109 | 57.9 | 96.3 | 87.7 | 26.6 |
| 0.5 | 919 | 57.8 | 55.9 | 69.2 | 21.4 |
| 1.0 | 52.5 | 60.6 | 36.6 | 49.9 | 12.2 |
| 1.5 | 46.8 | 46.9 | 16.0 | 36.6 | 17.8 |
| 2.0 | 37.7 | 33.4 | 16.7 | 29.3 | 11.1 |
| Animal Weight (kg) | 0.287 | 0.291 | 0.299 | 0.292 | 0.006 |
| Volume Dosed (mL) | 0.150 | 0.150 | 0.150 | 0.150 | 0.000 |
| Amount Dosed (mg/kg) | 5.23 | 5.15 | 5.02 | 5.13 | 0.11 |
| C_{max•}(ng/mL) | 139 | 72.7 | 108 | 107 | 33.2 |
| tₘₐₓ (hr) | 0.08 | 0.08 | 0.08 | 0.08 | 0.00 |
| tₘₐₓ (hr) | 2.09 | 1.16 | ND¹ | 1.63 | ND |
| AUCₗₐₛₜ (hr · ng/mL) | 134 | 105 | 85.0 | 108 | 24.8 |
| AUCₒₒ (hr · ng/mL) | 248² | 161² | ND¹ | 205 | ND |
| Dose Normalized Values³ | | | | | |
| AUCₗₐₛₜ (hr · kg · ng/mL/mg) | 25.7 | 20.4 | 16.9 | 21.0 | 4.41 |
| AUCₒₒ (hr · kg · ng/mL/mg) | 47.5² | 31.3² | ND¹ | 39.4 | ND |
| Bioavailability (%)⁴ | 1.33 | 1.06 | 0.88 | 1.09 | 0.23 |

Cₘₐₓ: Maximum plasma concentration;
tₘₐₓ: Time of maximum plasma concentration;
t_{1/2}: half-life, data points used for half-life determination are in bold;
AUCₗₐₛₜ: Area Under the Curve, calculated to the last observable time point;
AUCₒₒ: Area Under the Curve, extrapolated to infinity;
ND: Not Determined;
BLOQ: Below the limit of quantitation (10 ng/mL);
¹not determined due to terminal log linear phase not observed;
²AUC, is a greater than 25% extrapolation above its respective AUCₒₒ value;
³Dose normalized by dividing the parameter by the actual dose;
⁴Bioavailability determined by dividing the individual dose normalized intraduodenal AUCₗₐₛₜ values by the average dose normalized IV AUCₗₐₛₜ value from 11HAWAP1R1.

TABLE 11
Individual and Average Plasma Concentrations (ng/mL) and
Pharmacokinetic Parameters for Zanamivir After Intraduodenal
Administration in Male Sprague-Dawley Rats at 1.5 mg/animal
Intraduodenal (1.5 m animal; 50 μL PBS, 150 μL Blank Glycerol-2 hr)

| | Rat # | | | | |
|---|---|---|---|---|---|
| Time (hr) | 187 | 188 | 189 | Mean | SD |
| 0 (pre-dose) | BLOQ | BLOQ | BLOQ | ND | ND |
| 0.83 | 22.7 | 1.47 | 48.9 | 24.4 | 23.8 |
| 0.25 | 65.2 | 28.9 | 61.2 | 51.8 | 19.9 |
| 0.5 | 50.2 | 40.1 | 72.3 | 54.2 | 16.5 |
| 1.0 | 36.4 | 39.8 | 38.4 | 38.2 | 1.71 |
| 1.5 | 40.1 | 45.8 | 34.5 | 40.1 | 5.65 |
| 2.0 | 53.4 | BLOQ | 54.8 | 54.1 | ND |
| Animal Weight (kg) | 0.290 | 0.280 | 0.300 | 0.290 | 0.010 |
| Volume Dosed (mL) | 0.050 | 0.050 | 0.050 | 0.050 | 0.000 |
| Amount Dosed (mg/kg) | 5.17 | 5.36 | 5.00 | 5.18 | 0.18 |
| Cₘₐₓ (ng/mL) | 65.2 | 45.8 | 72.3 | 61.1 | 13.7 |
| tₘₐₓ (hr) | 0.25 | 1.50 | 0.50 | 0.75 | 0.66 |
| t_{1/2} (hr) | ND¹ | ND¹ | ND¹ | ND | ND |
| AUCₗₐₛₜ (hr · ng/mL) | 86.9 | 52.6 | 96.1 | 78.5 | 22.9 |
| AUC, (hr · ng/mL) | ND′ | ND¹ | ND¹ | ND | ND |
| Dose Normalized Values² | | | | | |
| AUCₗₐₛₜ (hr · kg · ng/mL/mg) | 16.8 | 9.81 | 19.2 | 15.3 | 4.89 |
| AUCₒₒ (hr · kg · ng/mL/mg) | ND¹ | ND¹ | ND¹ | ND | ND |
| Bioavailability (%)³ | 0.87 | 0.51 | 1.00 | 0.79 | 0.25 |

Cₘₐₓ: Maximum plasma concentration;
tₘₐₓ: Time of maximum plasma concentration;
t_{1/2}: half-life, data points used for half-life determination are in bold;
AUC_{Iast}: Area Under the Curve, calculated to the last observable time point;
AUCₒₒ: Area Under the Curve, extrapolated to infinity;
ND: Not Determined;
BLOQ: Below the limit of quantitation (10 ng/mL);
¹not determined due to terminal log linear phase not observed;
²Dose normalized by dividing the parameter by the actual dose;
³Bioavailability determined by dividing the individual dose normalized intraduodenal AUCₗₐₛₜ values by the average dose normalized AUCₗₐₛₜ value from 11HAWAP1R1.

TABLE 12
Individual and Average Plasma Concentrations (ng/mL) and
Pharmacokinetic Parameters for Zanamivir After Intraduodenal
Administration inMale Sprague-Dawley Rats at 1.5 mg/animal
Intraduodenal (1.5 mg/animal; 50 μL PBS,
50 μL Blank Capmul MCM L8-2 hr)

| | Rat # | | | | |
|---|---|---|---|---|---|
| Time (hr) | 190 | 191 | 192 | Mean | SD |
| 0 (pre dose) | BLOQ | BLOQ | BLOQ | ND | ND |
| 0.83 | 18.1 | 8.00 | 6.27 | 10.8 | 6.39 |
| 0.25 | 33.3 | 27.1 | 28.1 | 29.5 | 3.33 |
| 0.5 | 38.0 | 34.5 | 42.9 | 38.5 | 4.22 |
| 1.0 | 33.0 | 33.1 | 32.1 | 32.7 | 0.55 |
| 1.5 | 61.5 | 23.2 | 31.4 | 38.7 | 20.2 |
| 2.0 | 68.3 | 14.8 | 18.2 | 33.8 | 30.0 |
| Animal Weight (kg) | 0.296 | 0.280 | 0.285 | 0.287 | 0.008 |
| Volume Dosed (mL) | 0.050 | 0.050 | 0.050 | 0.050 | 0.000 |
| Amount Dosed (mg/kg) | 5.07 | 5.36 | 5.26 | 5.23 | 0.15 |
| Cₘₐₓ (ng/mL) | 68.3 | 34.5 | 42.9 | 48.6 | 17.6 |
| tₘₐₓ (hr) | 2.00 | 0.50 | 0.50 | 1.00 | 0.87 |
| t_{1/2} (hr) | ND¹ | 0.86 | ND² | 0.86 | ND |
| AUCₗₐₛₜ (hr · ng/mL) | 87.8 | 51.4 | 59.0 | 66.1 | 19.2 |
| AUCₒₒ (hr · ng/mL) | ND¹ | 69.8¹ | ND² | 69.8 | ND |
| Dose Normalized Values⁴ | | | | | |
| AUCₗₐₛₜ (hr · kg · ng/mL/mg) | 173 | 9.60 | 11.2 | 12.7 | 4.07 |
| AUCₒₒ (hr · kg · ng/mL/mg) | ND¹ | 13.0¹ | ND² | 13.0 | ND |
| Bioavailability (%)⁵ | 0.90 | 0.50 | 0.58 | 0.66 | 0.21 |

Cₘₐₓ: Maximum plasma concentration;
tₘₐₓ.: Time of maximum plasma concentration;
t_{1/2}: half-life, data points used for half-life determination are in bold;
AUCₗₐₛₜ: Area Under the Curve, calculated to the last observable time point;
AUCₒₒ: Area Under the Curve, extrapolated to infinity;
ND: Not Determined; Below the limit of quantitation (10 ng/mL);
¹not determined due to terminal log linear phase not observed;
²not determined due to correlation coefficient (R²) was less than 0.85;
³AUC,,, is a greater than 25% extrapolation above its respective AUC^{last} value;
⁴Dose normalized by dividing the parameter by the actual dose;
⁵Bioavailability determined by dividing the individual dose normalized intraduodenal AUC^{Iast} values by the average dose normalized IV AUC^{Iast} value from 11HAWAP1R1

Summary of pharmacokinetic results is presented in Table 13.

TABLE 13
Summary of Pharmacokinetic Parameters for Zanamivir from Different Formulations
after Intraduodenal Administration in Male Sprague-Dawley Rats at 1.5 mg/animal

| | Intraduodenal | | | | | | |
|---|---|---|---|---|---|---|---|
| | | | | | | | PBS |
| | | | | | PBS | PBS | (50 μL Blank |
| | Glycerol | Glycerol | Glycerol | | (50 μL Blank | (150 μL Blank | Capmul MCM |
| | 50 μL* | 100 μL | 150 μL | PBS* | Glycerol −2 hr)* | Glycerol −2 hr) | L8 −2 hr) |
| PK parameter | | | | | | | |
| Cₘₐₓ (ng/mL) | 948 | 76.1 | 107 | 134 | 77.4 | 61.1 | 48.6 |
| tₘₐₓ (hr) | 0.50 | 0.72 | 0.08 | 0.50 | 0.58 | 0.75 | 1.00 |
| t_{1/2} (hr) | 0.29 | 1.77 ^{a} | 1.63^{b} | 0.83 | 1.24 | ND | 0.86 ^{a} |
| AUCₗₐₛₜ (hr · ng/mL) | 754 | 96.4 | 108 | 162 | 112 | 78.5 | 66.1 |
| AUCₒₒ (hr · ng/mL) | 781 | [symbol] | [symbol] | [symbol] | 156 | ND | 69.8 |
| Dose Normalized Values¹ | | | | | | | |
| AUCₗₐₛₜ (hr · kg · ng/mL/mg) | 145 | 18.6 | 21.0 | 30.6 | 21.1 | 15.3 | 12.7 |
| AUCₒₒ(hr · kg · ng/mL/mg | 153 | 40.7 | 39.4 | 46.6 | 29.1 | ND | 13.0 |
| Bioavailability (%) | 7.53 | 0.97 | 1.09 | 1.59 | 1.10 | 0.79 | 0.66 |

Cₘₐₓ: Maximum plasma concentration;
tₘₐₓ: Time of maximum plasma concentration; to half-life;
AUCₗₐₛₜ: Area Under the Curve, calculated to the last observable time point;
AUCₒₒ: Area Under the Curve extrapolated to infinity;
¹Dose normalized by dividing the parameter by the actual dose;
*Data collected from 11HAWAP1R1 study;
^{a} n = 1,
^{b}n = 2. [symbol]
indicates data missing or illegible when filed

### Variation of Intraduodenal Enhancer and Zanamivir Levels on Absolute Bioavailability.

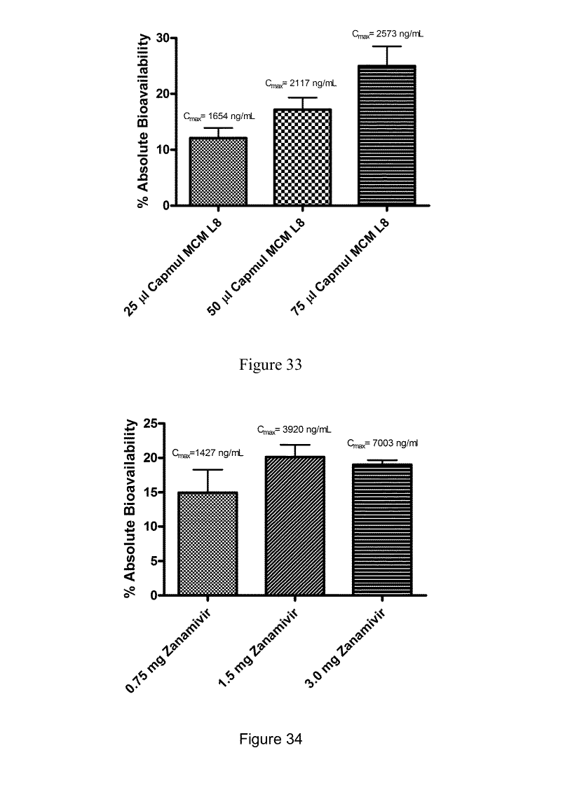

The effect of increasing intraduodenally administered Capmul MCM L8 at a fixed 1.5 mg zanamivir drug load on absolute bioavailability is shown in FIG. 33. A roughly linear increase in both absolute bioavailability of zanamivir and Cₘₐₓ are observed as Capmul MCM L8 amounts are increased 3-fold from 25 μL to 75 μL. These results demonstrate that enhancer amounts can be varied to optimize drug absorption and the associated pharmacokinetic parameters.

FIG. 34 summarizes the reciprocal results from varying zanamivir levels at a fixed 50 μL amount of Capmul MCM L8 after intraduodenal administration. Although there was only a modest difference in the absolute bioavailability of zanamivir as the dose varied 4-fold from 0.75 mg to 3.0 mg, there was a substantial impact on the resulting Cₘₐₓ. The Cₘₐₓ varied roughly proportionately to the drug load, with a short tₘₐₓ of 0.08, 0.08, and 0.14 hr for the 0.75 mg, 1.5 mg, and 3.0 mg zanamivir dosages, respectively. These results suggest that once the enhancer opened tight junctions to facilitate paracellular absorption, very rapid drug uptake occurred for a short duration, presumably due to only transient stimulation of the paracellular pathway.

### Example 7

### Determination of the Intraduodenal Bioavailability of Zanamivir from Different Formulations in Male Sprague-Dawley Rats

In this example, the bioavailability of Zanamivir was evaluated after intraduodenal doses in male Sprague-Dawley rats. The test compound was dosed at 1.5 mg/animal through intravenous and intraduodenal routes from normal saline and Capmul MCM L8 formulations, respectively. Plasma levels were determined by LC-MS/MS analysis. Pharmacokinetic parameters were estimated by a non-compartmental model using WinNonlin v5.2.1 software.

Following intravenous dosing at 1.5 mg/animal, average Cₘₐₓ values of 31194±3968 ng/mL were observed. The average clearance and volume of distribution were 0.614±0.038 L/hr/kg and 0.271±0.010 L/Kg, respectively. Half life was found to be 0.396±0.035 hours.

After intraduodenal dosing at 1.5 mg/animal from the Capmul MCM L8 (25 μL) formulation, Cₘₐₓ of 1654±645 ng/mL was reached at 5 min with average half-life of 0.453±0.053 hours. The overall percent bioavailability was found to be 12.1±3.12.

After intraduodenal dosing at 1.5 mg/animal from the Capmul MCM L8 (50 μL) formulation, Cₘₐₓ of 2117±510 ng/mL was reached at 5 min with average half-life of 0.410+0.050 hours. The overall percent bioavailability was found to be 17.2 3.77.

After intraduodenal dosing at 1.5 mg/animal from the Capmul MCM L8 (75 μL) formulation, Cₘₐₓ of 2573±750 ng/mL was reached between 5 and 15 min with average half-life of 0.415±0.063 hours. The overall percent bioavailability was found to be 25.0+6.09.

With increase in the vehicle dose volume from 25 μL to 75 μL, the ID bioavailability increased. There was significant difference (p<0.05) in the bioavailability observed between 25 μL and 75 μL volume dosed groups.

No adverse reactions were observed after intravenous and intraduodenal dosing of Zanamivir from different formulations in this study.

Each ID dose in groups 2-4 will be followed with a small air bubble (—10 4) and a flush of 125 4 of PBS to insure the dose is given in full. The volume of PBS used for cannulae flush will be maintained consistent across the animals and the treatment groups. The cannula will then be tied to help prevent the PBS remaining in the cannula from entering the duodenum.

The dosing solution was analyzed by LC-MS/MS. The measured dosing solution concentrations are shown in Table 14. Nominal dosing solution concentrations were used in all calculations. All concentrations are expressed as mg/mL of the free drug.

TABLE 14
Measured Dosing Solution Concentrations mg/mL

| | | | | Measured | |
|---|---|---|---|---|---|
| | | | | Dosing | |
| | | | Nominal | Solution | |
| Test | Route of | | Dosing | Concen- | |
| Com- | Adminis- | | Conc. | tration | % of |
| pound | tration | Vehicle | (mg/mL) | (mg/mL) | Nominal |
| Zanamivir | IV | Normal saline | 5.0 | 4.33 | 86.7 |
| | ID | Capmul MCM | 60.0 | 68.0 | 113 |
| | | L8 | 30.0 | 23.3 | 77.6 |
| | | | 20.0 | 20.8 | 104 |

Plasma samples were analyzed using the methods outlined in Appendix I. Plasma concentrations for Zanamivir are shown in Tables 15-18.

TABLE 15
Individual and Average Plasma Concentrations (ng/mL) and
Pharmacokinetic Parameters for Zanamivir After Intravenous
Administration in Male Sprague-Dawlev Rats at 1.5 mg/animal
Intravenous (1.5 mg/animal; Normal Saline)-

| | Rat # | | | | |
|---|---|---|---|---|---|
| Time (hr) | 954 | 955 | 956 | Mean | SD |
| 0 (pre-dose) | BLOQ | BLOQ | BLOQ | ND | ND |
| 0.033 | 20900 | 22900 | 20400 | 21400 | 1323 |
| 0.083 | 11000 | 12300 | 13500 | 12267 | 1250 |
| 0.25 | 11000 | 9620 | 11600 | 10740 | 1015 |
| 0.5 | 6270 | 5500 | 6450 | 6073 | 505 |
| 1.0 | 1560 | 1520 | 2190 | 1757 | 376 |
| 1.5 | 741 | 661 | 862 | 755 | 101 |
| 2.0 | 278 | 220 | 432 | 310 | 110 |
| Animal Weight | 0.275 | 0.286 | 0.277 | 0.279 | 0.006 |
| (kg) | | | | | |
| Volume Dosed | 0.30 | 0.30 | 0.30 | 0.30 | 0.00 |
| (mL) | | | | | |
| Amount Dosed | 5.45 | 5.24 | 5.42 | 5.37 | 0.11 |
| (mg/kg) | | | | | |
| Cₒ (ng/mL)¹ | 32061 | 34657 | 26863 | 31194 | 3968 |
| tₘₐₓ (hr)¹ | 0.0 | 0.0 | 0.0 | 0.0 | 0.0 |
| T_{1/2} (hr) | 0.402 | 0.359 | 0.427 | 0.396 | 0.035 |
| CL (L/hr/kg) | 0.632 | 0.640 | 0.571 | 0.614 | 0.038 |
| Vₛₛ (L/kg) | 0.276 | 0.260 | 0.278 | 0.271 | 0.010 |
| AUCₗₐₛₜ | 8460 | 8076 | 9230 | 8589 | 587 |
| (hr · ng/mL) | | | | | |
| AUCₒₒ | 8627 | 8195 | 9485 | 8769 | 657 |
| (hr · ng/mL) | | | | | |
| Dose Normalized | | | | | |
| Values³ | | | | | |
| AUCₗₐₛₜ | 1551 | 1540 | 1704 | 1598 | 91.9 |
| (hr · kg · | | | | | |
| ng/mL/mg) | | | | | |
| AUCₒₒ | 1583 | 1564 | 1750 | 1632 | 102 |
| (hr · kg · | | | | | |
| ng/mL/mg) | | | | | |

Co: Maximum plasma concentration extrapolated to t = 0;
tₘₐₓ: Time of maximum plasma concentration;
t_{1/2}: half-life, data points used for half-life determination are in bold;
CL: Clearance;
Vₛₛ: Steady state volume of distribution;
AUC_{Iast}: Area Under the Curve, calculated to the last observable time point;
AUCₒₒ: Area Under the Curve, extrapolated to infinity;
ND: Not Determined;
BLOQ: Below the limit of quantitation (10 ng/mL);
¹Extrapolated to t = 0;
²Dose normalized by dividing the parameter by the actual dose.

TABLE 16
Individual and Average Plasma Concentrations (ng/mL) and
Pharmacokinetic Parameters for Zanamivir After Intraduodenal (Bolus)
Administration in Male Sprague-Dawley Rats at 1.5 mg/animal
Intraduodenal (bolus) (1.5 mg/animal; 25 μL Capmul MCM L8)

| | Rat # | | | | |
|---|---|---|---|---|---|
| Time (hr) | 957 | 958 | 959 | Mean | SD |
| 0 (pre-dose) | BLOQ | BLOQ | BLOQ | ND | ND |
| 0.83 | 1960 | 2090 | 913 | 1654 | 645 |
| 0.25 | 1500 | 1380 | 655 | 1178 | 457 |
| 0.5 | 916 | 950 | 647 | 838 | 166 |
| 1.0 | 387 | 378 | 259 | 341 | 71.4 |
| 1.5 | 145 | 143 | 119 | 136 | 14.5 |
| 2.0 | 70.7 | 78.2 | 66.8 | 71.9 | 5.79 |
| Animal Weight (kg) | 0.284 | 0.271 | 0.280 | 0.278 | 0.007 |
| Volume Dosed (mL) | 0.025 | 0.025 | 0.025 | 0.025 | 0.000 |
| Amount Dosed (mg/kg) | 5.28 | 5.54 | 5.36 | 5.39 | 0.13 |
| Cₘₐₓ (ng/mL) | 1960 | 2090 | 913 | 1654 | 645 |
| tₘₐₓ (hr) | 0.08 | 0.08 | 0.08 | 0.08 | 0.00 |
| t_{1/2} (hr) | 0.408 | 0.440 | 0.512 | 0.453 | 0.053 |
| AUCₗₐₛₜ (hr · ng/mL) | 1185 | 1185 | 699 | 1023 | 281 |
| AUCₒₒ (hr · ng/mL) | 1224 | 1232 | 747 | 1068 | 278 |
| Dose Normalized Values¹ | | | | | |
| AUCₗₐₛₜ (hr · kg · ng/mL/mg) | 224 | 214 | 130 | 190 | 51.5 |
| AUCₒₒ (hr · kg · ng/mL/mg) | 232 | 222 | 139 | 198 | 50.9 |
| Bioavailability (%)² | 14.2 | 13.6 | 8.53 | 12.1 | 3.12 |

Cₘₐₓ: Maximum plasma concentration;
tₘₐₓ: Time of maximum p asma concentration;
t_{1/2}: half-life, data points used for half-life determination are in bold;
Area Under the Curve, calculated to the last observable time point;
AUCₒₒ,: Area Under the Curve, extrapolated to infinity;
ND: Not Determined;
BLOQ: Below the limit of quantitation (10 ng/mL);
¹Dose normalized by dividing the parameter by the actual dose;
²Bioavailability determined by dividing the individual dose normalized intraduodenal AUC_{inf} values by the average dose normalized IV AUC_{inf} value.

TABLE 17
Individual and Average Plasma Concentrations (ng/mL) and
Pharmacokinetic Parameters for Zanamivir After Intraduodenal (Bolus)
Administration in Male Sprague-Dawley Rats at 1.5 mg/animal
Intraduodenal (bolus) (1.5 mg/animal; 50 μL Capmul MCM L8)

| | Rat # | | | | |
|---|---|---|---|---|---|
| Time (hr) | 960 | 961 | 962 | Mean | SD |
| 0 (pre dose) | BLOQ | BLOQ | BLOQ | ND | ND |
| 0.83 | 2620 | 2130 | 1600 | 2117 | 510 |
| 0.25 | 2060 | 1400 | 1270 | 1577 | 424 |
| 0.5 | 1250 | 985 | 774 | 1003 | 239 |
| 1.0 | 695 | 708 | 551 | 651 | 87.1 |
| 1.5 | 216 | 299 | 217 | 244 | 47.6 |
| 2.0 | 103 | 158 | 99.5 | 120 | 32.8 |
| Animal Weight (kg) | 0.294 | 0.265 | 0.272 | 0.277 | 0.015 |
| Volume Dosed (mL) | 0.050 | 0.050 | 0.050 | 0.050 | 0.000 |
| Amount Dosed (mg/kg) | 5.10 | 5.66 | 5.51 | 5.43 | 0.29 |
| Cₘₐₓ (ng/mL) | 2620 | 2130 | 1600 | 2117 | 510 |
| tₘₐₓ (hr) | 0.08 | 0.08 | 0.08 | 0.08 | 0.00 |
| t_{1/2} (hr) | 0.363 | 0.462 | 0.405 | 0.410 | 0.050 |
| AUCₗₐₛₜ (hr · ng/mL) | 1707 | 1470 | 1164 | 1447 | 272 |
| AUCₒₒ (hr · ng/mL) | 1757 | 1572 | 1220 | 1516 | 273 |
| Dose Normalized Values¹ | | | | | |
| AUCₗₐₛₜ (hr · kg · ng/mL/mg) | 335 | 260 | 211 | 268 | 62.2 |
| AUCₒₒ (hr · kg · ng/mL/mg) | 344 | 278 | 221 | 281 | 61.6 |
| Bioavailability (%)² | 21.1 | 17.0 | 13.6 | 17.2 | 3.77 |

Cₘₐₓ: Maximum plasma concentration;
tₘₐₓ: Time of maximum plasma concentration;
t_{1/2}: half-life, data points used for half-life determination are in bold;
AUCₗₐₛₜ: Area Under the Curve, calculated to the last observable time point;
AUCₒₒ: Area Under the Curve, extrapolated to infinity;
ND: Not Determined;
BLOQ: Below the limit of quantitation (10 ng/mL);
¹Dose normalized by dividing the parameter by the actual dose;
²Bioavailability determined by dividing the individual dose normalized intraduodenal AUCmf values by the average dose normalized IV AUC_{inf} value.

TABLE 18
Individual and Average Plasma Concentrations (ng/mL) and
Pharmacokinetic Parameters for Zanamivir After Intraduodenal (Bolus)
Administration in Male Sprague-Dawley Rats at 1.5 mg/animal
Intraduodenal (bolus) (1.5 mg/animal; 75 μL Capmul MCM L8)

| | Rat # | | | | |
|---|---|---|---|---|---|
| Time (hr) | 963 | 964 | 965 | Mean | SD |
| 0 (pre dose) | BLOQ | BLOQ | BLOQ | ND | ND |
| 0.83 | 2940 | 1690 | 3070 | 2567 | 762 |
| 0.25 | 2560 | 1710 | 2890 | 2387 | 609 |
| 0.5 | 1820 | 1130 | 2200 | 1717 | 542 |
| 1.0 | 748 | 807 | 929 | 828 | 92.3 |
| 1.5 | 403 | 273 | 372 | 349 | 67.9 |
| 2.0 | 180 | 121 | 160 | 154 | 30.0 |
| Animal Weight (kg) | 0.286 | 0.273 | 0.280 | 0.280 | 0.007 |
| Volume Dosed (mL) | 0.075 | 0.075 | 0.075 | 0.075 | 0.000 |
| Amount Dosed (mg/kg) | 5.24 | 5.49 | 5.36 | 5.37 | 0.13 |
| Cₘₐₓ (ng/mL) | 2940 | 1710 | 3070 | 2573 | 750 |
| tₘₐₓ (hr) | 0.08 | 0.25 | 0.08 | 0.14 | 0.10 |
| t_{1/2} (hr) | 0.487 | 0.365 | 0.394 | 0.415 | 0.063 |
| AUCₗₐₛₜ (hr · ng/mL) | 2204 | 1562 | 2501 | 2089 | 480 |
| AUCₒₒ (hr · ng/mL) | 2334 | 1622 | 2591 | 2183 | 502 |
| Dose Normalized Values¹ | | | | | |
| AUCₗₐₛₜ (hr · kg · ng/mL/mg) | 420 | 284 | 467 | 390 | 94.9 |
| AUCₒₒ (hr · kg · ng/mL/mg) | 445 | 296 | 483 | 408 | 99.4 |
| Bioavailability (%)² | 27.3 | 18.1 | 29.6 | 25.0 | 6.09 |

Cₘₐₓ: Maximum plasma concentration;
tₘₐₓ: Time of maximum plasma concentration;
t_{1/2}: half-life, data points used for half-life determination are in bold;
AUCₗₐₛₜ: Area Under the Curve, calculated to the last observable time point;
AUCₒₒ: Area Under the Curve, extrapolated to infinity;
ND: Not Determined;
BLOQ: Below the limit of quantitation (10 ng/mL);
¹Dose normalized by dividing the parameter by the actual dose;
²Bioavailability determined by dividing the individual dose normalized intraduodenal AUC_{inf} values by the average dose normalized IV AUC_{inf} value.

Summary of pharmacokinetic results is presented in Table 19.

TABLE 19
Summary of Pharmacokinetic Parameters for Zanamivir from
Different Formulations after IntraduodenalAdministration in Male
Sprague-Dawley Rats at 1.5 mg/animal

| | Intraduodenal (Caprnul MCM L8) | | |
|---|---|---|---|
| PK Parameter | 25 μL | 50 μL | 75 μL |
| Cₘₐₓ (ng/mL) | 1654 | 2117 | 2573 |
| tₘₐₓ (hr) | 0.08 | 0.08 | 0.14 |
| t_{1/2} (hr) | 0.453 | 0.410 | 0.415 |
| AUCₗₐₛₜ (hr · ng/mL) | 1023 | 1447 | 2089 |
| AUCₒₒ (hr · ng/mL) | 1068 | 1516 | 2183 |
| Dose Normalized Values¹ | | | |
| AUCₗₐₛₜ (hr · kg · ng/mL/mg) | 190 | 268 | 390 |
| AUCₒₒ (hr · kg · ng/mL/mg) | 198 | 281 | 408 |
| Bioavailability (%)² | 12.1 | 17.2 | 25.0 |

Cₘₐₓ: Maximum plasma concentration;
tₘₐₓ: Time of maximum plasma concentration; t_{1/2}: half-life;
AUCᵢₐ,,: Area Under the Curve, calculated to the last observable time point;
AUCₒₒ: Area Under the Curve, extrapolated to infinity;
¹Dose normalized by dividing the parameter by the actual dose;
²Bioavailability determined by dividing the individual dose normalized intraduodenal AUC_{inf} values by the average dose normalized IV AUC_{inf} value.

### Example 8

### Summary of Caco-2 Permeability Data

To summarize the results set forth above, Caco-2 membrane permeability of zanamivir as a function of the vehicle used therefore (i.e., PBS control, 5% glycerol or 0.25% Capmul MCM L8) is presented in FIGS. 31A, 31B and 31C.

### Example 9

### Summary of Absolute Bioavailability of Zanamivir

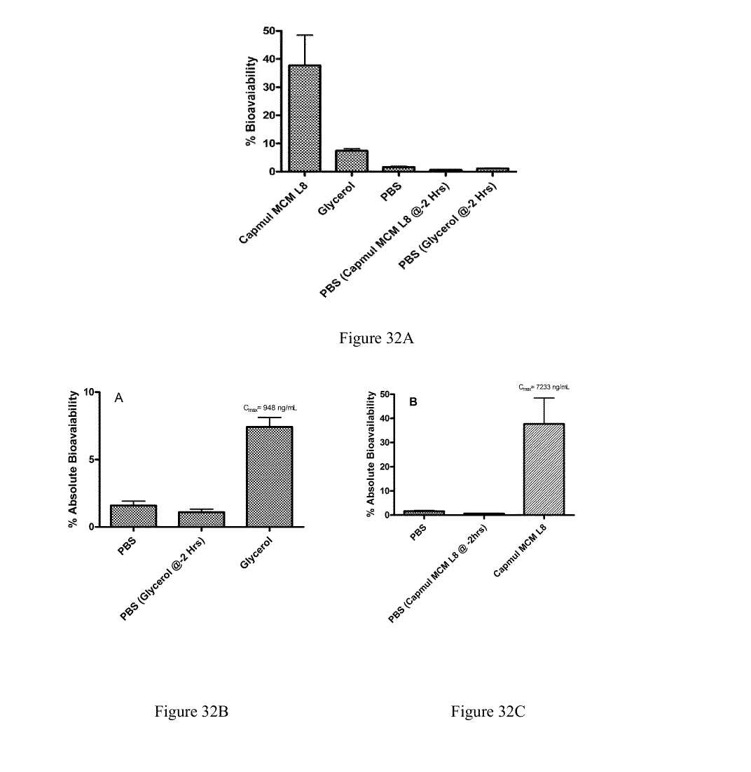

The absolute bioavailability of 1.5 mg zanamivir administered intraduodenally with 50 μl of vehicle, is presented in FIGS. 32A and 32B.

### Example 10

### Variation of Intraduodenal Enhancer and Zanamivir Levels on Absolute Bioavailability

The effect of increasing intraduodenally administered Capmul MCM L8 at a fixed 1.5 mg zanamivir drug load on absolute bioavailability is shown in FIG. 34. A roughly linear increase in both absolute bioavailability of zanamivir and Cₘₐₓ are observed as Capmul MCM L8 amounts are increased 3-fold from 25 μL to 75 μL. These results demonstrate that enhancer amounts can be varied to optimize drug absorption and the associated pharmacokinetic parameters.

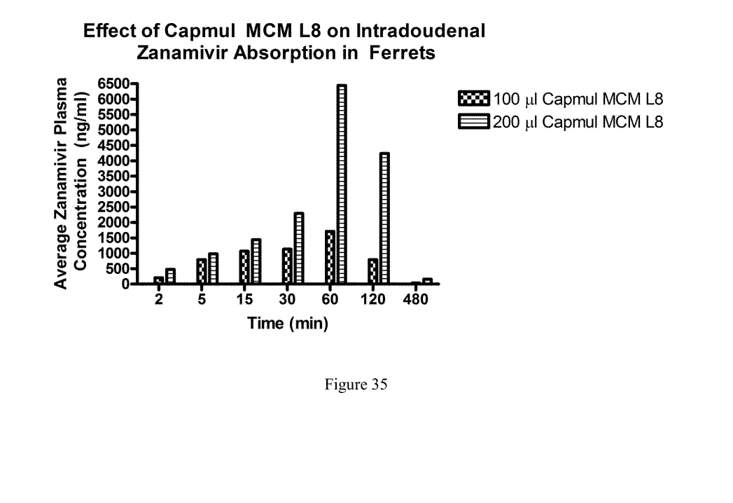

FIG. 35 summarizes the reciprocal results from varying zanamivir levels at a fixed 50 μL amount of Capmul MCM L8 after intraduodenal administration. Although there was only a modest difference in the absolute bioavailability of zanamivir as the dose varied 4-fold from 0.75 mg to 3.0 mg, there was a substantial impact on the resulting Cₘₐₓ. The Cₘₐₓ varied roughly proportionately to the drug load, with a short tₘₐₓ of 0.08, 0.08, and 0.14 hr for the 0.75 mg, 1.5 mg, and 3.0 mg zanamivir dosages, respectively. These results suggest that once the enhancer opened tight junctions to facilitate paracellular absorption, very rapid drug uptake occurred for a short duration, presumably due to only transient stimulation of the paracellular pathway.

### Example 11

### Proposed Initial Human Pharmacokinetic Trial

This is a prophetic example. To be effective, a proposed enteric-coated zanamivir oral dosage form should contain an adequate amount of a permeability enhancer to impact either the paracellular or transcellular transport pathways, or both. Once such a condition has been identified, the amount of zanamivir can be appropriately scaled to achieve the desired blood level. For example, the amount of permeability enhancer should take into account the volume of a human duodenum: 750-1000 mg and 1500-2000 mg of enhancer should roughly correspond to the dose at the lower and upper ranges, respectively, of the proportionate volume of the human duodenum.

An initial human PK trial should be designed to test the utility of both Capmul® MCM L8 and glycerol. A four- or five-way crossover protocol utilizing enteric-coated softgels is envisioned. This involves dosing subjects with either one or two softgels in separate arms and examining the PK data to determine if the zanamivir blood levels are dose proportional. Zanamivir dose proportionality would indicate a near saturating effect from the lower dose of the permeability enhancer used. Alternatively, separate dosage forms can be manufactured for each arm wherein zanamivir is kept constant and two amounts of permeability enhancer is used. The following arms are proposed to both test permeability enhancer function and to limit the number of dosage forms that must be manufactured.

Arm 1: 150 mg zanamivir, 765 mg Capmul MCM L8 in a single dosage form (765 mg of Capmul MCM L8 is the highest currently approved amount on the FDA inactive ingredient list.)

Arm 2: 300 mg zanamivir, 1530 mg Capmul MCM L8 dosed as two gelcaps used in Arm 1.

Arm 3: 150 mg zanamivir, 1000 mg glycerol in a single dosage form (although 223.8 mg of glycerol is the highest currently approved amount, its safety and use as a food additive should not present a significant regulatory barrier for increasing that limit.)

Arm 4: 300 mg zanamivir, 2000 mg glycerol dosed as two gelcaps used in Arm 3.

Arm 5: 150 mg or 300 mg zanamivir plus inert filler in a single dosage form (this is an optional negative control arm included to scale the impact of the permeability enhancers. It may be unnecessary depending on prior clinical experience with oral zanamivir.)

It is anticipated that results from this trial will provide important information to demonstrate the potential to deliver zanamivir orally and to use as a guide in defining an optimized formulation and zanamivir drug load.

Although the invention is illustrated and described herein with reference to specific embodiments, the invention is not intended to be limited to the details shown. Rather, various modifications may be made in the details within the scope and range of equivalents of the claims without departing from the invention.

That which is claimed is:

1. A composition comprising:
zanamivir, and
a permeability enhancer,
wherein the composition increases the amount of zanamivir which is transported across the Caco-2 cell membrane by at least 150% relative to the amount of zanamivir which is transported across the Caco-2 cell membrane in the absence of the permeability enhancer.

2. The composition of claim 1 for treating or preventing influenza infection.

3. A composition comprising:
zanamivir, and
a permeability enhancing amount of a permeability enhancer.

4. A composition for treating or preventing influenza infection, said composition comprising:
zanamivir, and
a permeability enhancer,
wherein the composition increases the bioavailability of zanamivir in a subject to which said composition is administered by at least 10% relative to the bioavailability of zanamivir in a subject to which said composition is administered in the absence of the permeability enhancer.

5. The composition of claim 1, wherein said permeability enhancer is a fatty acid, or a salt or ester thereof.

6. The composition of claim 5, wherein said fatty acid is a C8 to C10 acid, as well as a salt or ester thereof.

7. The composition of claim 1 wherein the permeability enhancer is present in the amount of at least 0.1 wt % of the combined weight of enhancer and zanamivir.

8. The composition of claim 7 wherein the permeability enhancer is present in the amount of no greater than 99 wt % of the combined weight of enhancer and zanamivir.

9. The composition of claim 1, wherein the composition increases the amount of zanamivir which is transported across the Caco-2 cell membrane by at least 250% relative to the amount of zanamivir which is transported across the Caco-2 cell membrane in the absence of the permeability enhancer.

10. The composition of claim 1, further comprising an enteric coating thereon.

11. An oral dosage form comprising the composition of claim 1, wherein the composition comprises a therapeutically effective amount of zanamivir and a permeability enhancing amount of said permeability enhancer.

12. A unit dosage form comprising a single use dosage of the composition of claim 1, wherein the composition comprises a therapeutically effective amount of zanamivir and a permeability enhancing amount of said permeability enhancer.

13. A method of treating or preventing influenza infection, said method comprising administering a composition according to claim 1 to a subject in need thereof.

14. Use of a composition according to claim 1 in the preparation of a medicament for treating or preventing influenza infection.

15. A kit comprising a composition according to claim 1, and directions for the administration thereof to a subject in need thereof.
